(19) United States
(12) Patent Application Publication
(10) Pub. No.: US 2020/0316207 A1
(43) Pub. Date: Oct. 8, 2020

(54) POLYMERIC BILE ACID NANOPARTICLES AS ANTI-INFLAMMATORY AGENTS

(71) Applicant: Yale University, New Haven, CT (US)

(72) Inventors: Jung Seok Lee, New Haven, CT (US); Tarek M. Fahmy, Middlefield, CT (US); Dongin Kim, Glastonbury, CT (US)

(21) Appl. No.: 16/906,695

(22) Filed: Jun. 19, 2020

Related U.S. Application Data
(63) Continuation-in-part of application No. 15/757,608, filed on Mar. 5, 2018.
(60) Provisional application No. 62/214,648, filed on Sep. 4, 2015.

(51) Int. Cl. A61K 47/28; A61K 9/51; A61K 35/413; A61P 29/00

(52) U.S. Cl. CPC A61K 47/28; A61K 9/5153; A61K 9/0053; A61P 29/00; A61K 35/413

(57) ABSTRACT

Polymeric poly(bile acid) (pBA) nanoparticles have enhanced avidity and affinity to bile acid receptors and are effective anti-inflammatory agents. Oral delivery results in local accumulation and retention in the pancreas, liver, and colon as well as in systemic delivery of the nanoparticles. The nanoparticles are effective in alleviating inflammation and are useful as anti-inflammatory agents to treat inflammatory diseases of the organs. The nanoparticles provide a therapeutic and prophylactic benefit via the TGR5 pathway when used alone, or a more than additive benefit when used in combination with immunosuppressant(s). The nanoparticles induce immune tolerance in autoimmune diseases and are useful therapeutics for treating inflammatory and autoimmune diseases.

### CROSS-REFERENCE TO RELATED APPLICATIONS

This application is a continuation-in-part of U.S. application Ser. No. 15/757,608, filed Mar. 5, 2018, entitled “Polymeric Bile Acid Nanocompositions Targeting the Pancreas and Colon”, which is a National Phase application under 35 U.S.C. § 371 of International Application No. PCT/US2016/050291, filed Sep. 2, 2016, which claims priority to and benefit of U.S. Provisional Application No. 62/214,648 filed Sep. 4, 2015, by Tarek Fahmy, Jung Seok Lee, and Dongin Kim, which are hereby incorporated herein by reference in their entirety.

### STATEMENT REGARDING FEDERALLY SPONSORED RESEARCH

This invention was made with government support under 0747577 awarded by National Science Foundation and under AI056363, CA199004, and CA026412 awarded by National Institutes of Health. The government has certain rights in the invention.

### FIELD OF THE INVENTION

The invention is generally directed to polymeric bile acid nanocompositions which are orally administered for systemic delivery to suppress local pro-inflammatory immunity, even in the absence of antiinflammatory agent.

### BACKGROUND OF THE INVENTION

Numerous diseases and disorders are characterized by inflammation. Inflammation is a vital part of the immune system's response to injury and infection, signaling the immune system to heal and repair damaged tissue, as well as defend itself against foreign invaders, such as viruses and bacteria. Without inflammation as a physiological response, wounds would fester, and infections could become deadly. However, if the inflammatory process goes on for too long or if the inflammatory response occurs in places where it is not needed, it can become problematic. Chronic inflammation has been linked to certain diseases including heart disease and stroke, and may also lead to autoimmune disorders, such as rheumatoid arthritis and lupus.

Acute inflammation occurs after a cut on the knee, a sprained ankle or a sore throat. It is a short-term response with localized effects. The characteristic signs of acute inflammation include redness, swelling, heat and sometimes pain and loss of function. During acute inflammation, cytokines are released by the damaged tissue to recruit immune cells. Prostaglandins create blood clots to heal damaged tissue, triggering pain and fever as part of the healing process. As the body heals, the acute inflammation gradually subsides.

Unlike acute inflammation, chronic inflammation can have long-term and whole-body effects. Chronic inflammation is also called persistent, low-grade inflammation because it produces a steady, low-level of inflammation throughout the body, as judged by a small rise in immune system markers found in blood or tissue. This type of systemic inflammation can contribute to the development of disease.

Low levels of inflammation can be triggered by a perceived internal threat, even when disease or injury is not present, signaling the immune system to respond. Immune cells attracted to the area of inflammation may start attacking internal organs or other healthy tissues and cells. Chronic inflammation has been linked to heart disease and stroke. One theory suggests that when inflammatory cells stay too long in blood vessels, they promote the buildup of plaque. Cancer is another disease linked with chronic inflammation. Over time, chronic inflammation can cause DNA damage and lead to some forms of cancer. Autoimmune diseases such as rheumatoid arthritis, multiple sclerosis, psoriasis, and lupus erythematosus are also characterized by chronic inflammation.

Currently, there are no prescription drugs that specifically target chronic inflammation, although some drugs have been shown to alleviate symptoms of specific diseases associated with chronic inflammation either non-specifically, or specifically in the case of some of the autoimmune disorders. Non-steroidal anti-inflammatory drugs (NSAIDs) including aspirin, naproxen (Aleve) and ibuprofen (Advil and Motrin). NSAIDs work by blocking the enzyme cyclooxygenase, which produces prostaglandins, which promotes inflammation. Corticosteroids, such as cortisone and prednisone, may be prescribed for inflammatory conditions, such as asthma and arthritis. They may help suppress inflammation, but these drugs also carry a risk of side effects.

With few options available, there is a need for anti-inflammatory agents in general, as well as a need for anti-inflammatory agents which can be used for acute or chronic inflammation in specific diseases, having few to no side effects or toxicity.

Therefore, it is an object of the present invention to provide anti-inflammatory polymeric particles.

It is a further object of the present invention to provide an oral antiinflammatory composition.

It is still another object of the present invention to provide anti-inflammatory polymeric particles which preferentially target specific organs or regions of the body.

### SUMMARY OF THE INVENTION

Anti-inflammatory polymeric nanoparticles are formed of bile acid esterified polymers (pBA) having a molecular weight between about 800-1,000 (two monomers) and 240,000 Dalton (Da) (approximately 400 monomers), although they may have much larger molecular weights. The bile acid ester polymers are typically formed of one or more polymeric ursodeoxycholic acid (pUDCA), polymeric lithocholic acid (pLCA), polymeric deoxycholic acid (pDCA), polymeric chenodeoxycholic acid (pCDCA), and polymeric cholic acid (pCA). The bile acid ester polymers may be linear and/or branched polymers. References to pUDCA are generally applicable to other bile acid ester polymers. The nanoparticles formed of pBA may have diameters between 60 nm and 600 nm, more preferably between 100 nm and 400 nm, with a typical average geometric diameter of 350 nm. The polymeric nanoparticles may include other biocompatible polymer, as blends or as copolymers. In some embodiments, the nanoparticles are formed of pUDCA having a molecular weight between about 800 and 5,000 Da and having between about two and 20 UDCA monomeric units per polymer.

Typically, the bile acid ester polymers form a surface on the nanoparticles containing between 100 and 5000 bile acid monomeric units. The nanoparticles typically have at least 1.5 fold greater affinity, and up to about 50 fold greater affinity, to bile acid receptors than the respective monomers forming the bile acid ester polymers. The bile acid receptors include the G protein-coupled bile acid receptor 1 (GPBAR1 or Takeda G-protein receptor 5 (TGR5)) and the Farnesoid-X-Receptor (FXR). These receptors are placed at the interface of the host immune system with the intestinal microbiota and are highly represented in cells of innate immunity such as intestinal and liver macrophages, dendritic cells and natural killer T cells are generally on the surface of innate immune cells, such as macrophages.

The empty nanoparticles bind to the bile acid receptors to activate the anti-inflammatory responses from the innate immune cells. One or more symptoms or treating an inflammatory and/or an autoimmune disease in a subject is treated by orally administering to the subject a formulation containing an effective amount of the nanoparticles. Typically, the nanoparticles distribute to internal organs, such as the heart, kidneys, spleen, lungs, liver, colon, and pancreas, following oral administration. This distribution is typically mediated by particles' intestinal transport and permeation through intestinal epithelium assisted by macrophage engulfment (by binding to TGR-5, endocytosis, exocytosis) and enterohepatic circulation (gall bladder accumulation and pancreatic ductal entry), in the absence of tissue- or organ-specific targeting agent.

The inflammatory and/or an autoimmune diseases may be type 1 diabetes, type 2 diabetes, pancreatitis, systemic lupus erythematous, or rheumatoid arthritis. A method of treating type 1 diabetes is also described and includes orally administering to a subject in need thereof a formulation containing an effective amount of pBA nanoparticles alone.

The methods typically include administering the formulation for a period of at least one week, at least two weeks, or at least three weeks. The formulation may be administered three times a week, two times a week, or once or twice a day. Following treatment, the subject typically maintains healthy blood glucose for at least about three days, about five days, about one week, about two weeks, about one month, or more, after cessation of administering the formulation. The subject typically shows an increase in the number of regulatory T cells (Treg) relative to a control. The subject typically develops a tolerogenic phenotype.

Typical doses for treating inflammatory and/or autoimmune diseases include between 0.1 mg/kg and 1000 mg/kg, such as between about 0.4 mg/Kg and about 400 mg/Kg, between about 50 mg/Kg and 1000 mg/Kg, or between about 100 mg/Kg and 500 mg/Kg.

Methods of making NPs using self-assembly and aggregation of bile acid have been developed. Two methods for making the bile acid assemblies include fabrication of branched polymeric bile acid units (as opposed to linear chains), and encapsulation through guest/host interactions in cavities that form with such branched building blocks; and supramolecular self-assembly via fluorinated bile acid units. Fluorination introduces a “fluorophobic effect.” This is distinctly different from hydrophobic or hydrophilic interactions, and results in self-assembly into a complex larger structure without the need for special formulation.

The NPs can exhibit therapeutic and/or prophylactic effects on inflammation (e.g., for treating autoimmune diseases) and/or metabolic regulation (e.g., for controlling blood glucose level and weight). It is believed this is due to the pBA, and more preferably, pUDCA, binding to the TGR5 bile acid receptor. The binding avidity and affinity of NPs to the bile acid receptors is enhanced when compared to those of the respective bile acid monomers. The enhanced avidity and affinity is due to the polymerization of bile acids and surface properties of NPs exposing between 100 and 5000 bile acid monomeric units. This permits the use of NPs as therapeutics with increased potency and efficacy when compared to the use of bile acid monomers.

PBAs binding bile acid receptors activates an intracellular program that facilitates endogenous insulin secretion, energy metabolism, endogenous insulin receptor expression, and a host of other functions such as reduction in reactive oxygen species and reduction in pro-inflammatory signaling. In the embodiments where the particles encapsulate insulin, this anti-inflammatory effect on cells happens before the insulin is released from the particles and binds its receptors and regulates glucose. Therefore, the bile acid particles naturally mimic the physiologic process. As shown in the Examples, the NPs are therapeutic with broad-spectrum properties that manage T1D in the short-term and function to reverse pathology and restore endogenous insulin secretion and regulatory immunity in the long-term.

### BRIEF DESCRIPTION OF THE DRAWINGS

FIGS. 1A-1J are schematics of polymeric BAs (pBA) formulated into NPs under emulsion conditions. BAs (cholic acid (CA), chenodeoxycholic acid (CDCA), deoxycholic acid (DCA), lithocholic acid (LCA), and ursodeoxycholic acid (UDCA)) are shown in FIGS. 1A-1E.

FIGS. 1F-1J show (1) Esterification of carbon-24 position on monomeric BAs results in hydrolysable ester bonded BAs (pBAs). The schematic of the polymerization step shows the location of the polymer-forming reactive end groups. (2) Emulsification of pBAs in the presence of drug yields (3) drug entrapped in solid pBA NPs with an average diameter of 344.3±4.7 nm.

FIG. 2 is a schematic of bond correlations in pUDCA revealed by Key HMBC and COSY NMR spectra. Given the intensities of ¹H NMR signals which provide information on the relative number of protons, NMR data of pUDCA reveals that two hydroxyl substituents at C-3 and C-7 are esterified with 2.5:1 molar ratio during the polymerization process.

FIGS. 3A and 3B are graphs showing organ level biodistribution of orally ingested NPs (500 mg/kg): poly(cholic acid) (pCA), poly(lithocholic acid) (pLCA), poly(deoxycholic acid) (pDCA), poly(chenodeoxycholic acid) (pCDCA), poly(ursodeoxycholic acid) (pUDCA), and control poly(lactic-co-glycolic acid) (PLGA) (FIG. 3A), and dose dependent biodistribution. Mice were fasted then orally gavaged with DIR-encapsulating pBA NPs at 50, 100, and 500 mg/kg (FIG. 3B)

FIGS. 4A-4N are graphs showing properties of polymer bile acids (pBAs) in vitro and in vivo.

FIG. 4A is a comparison between pUDCA and control PLGA NPs in the biodistribution in non-gastrointestinal organs.

FIG. 4B shows dye-independent localization of NP in the pancreas. Pancreatic accumulation of NPs was quantitated when coumarin 6 was used as a tracer, confirming that level of pancreatic accumulation of NP was independent of the physiochemical properties of the loaded agent, but dependent on the particle composition. Free coumarin was dispersed in 1% tween 20 in saline.

FIG. 4C graphs the cytotoxicity of NPs (1 mg/mL) in Caco-2 cells (10⁴ cells/well) and BMMs (10⁴ cells/well) measured using a CELLTITER-BLUE® Cell Viability Assay (Promega Co.) after incubation at 37° C. for 24 h.

FIG. 4D shows a decrease in the interferon gamma (IFNγ) level from OT-II CD4+ T cells when OT-II T cells were cocultured with pUDCA-treated dendritic cells (DCs) that were stimulated by lipopolysaccharide (LPS) and ovalbumin (OVA).

FIG. 4E shows the impact of pUDCA on secretion of pro-inflammatory cytokine, IL-6, from macrophages.

FIG. 4F shows particle stability evaluated by measuring particle sizes over time in simulated stomach conditions (citrate buffer solution, pepsin 10 mg/mL, pH 2.0, 37° C.).

FIG. 4G graphs the bioactivity of released insulin from pUDCA. The released insulin from pUDCA at 3 or 24 h was incubated with CHO INSR cells for 1 h and pAkt was measured by ELISA. The pAkt production from CHO INSR cells that were incubated with fresh or denatured insulin was measured to calculate % bioactivity. The average bioactivity of released INS was 87.3% of fresh insulin.

FIG. 4H shows the pPermeability of NPs through a layer of Caco-2 cells on transwell filters.

FIG. 4I depicts pancreatic trafficking with and without macrophage depletion. B6 mice were depleted of macrophages and treated with DIR-loaded pUDCA NPs by oral gavage (500 mg/kg, 250 μL). Clodrosome (Clodronate-containing liposomes, 100 mg/kg, IP) was used to deplete macrophages. Pancreata were harvested at 4 h post gavage and imaged.

FIG. 4J shows CD11c-F4/80+ macrophages associated with coumarin 6-loaded pUDCA NPs in pancreas, liver, lungs, and spleen in mice as acquired using a flow cytometer at 4 h post oral ingestion.

FIG. 4K depicts competitive binding of pUDCA and UDCA to TGR5 on macrophages at 4° C.

FIG. 4L graphs the rate of endocytosis 37° C. and exocytosis at 4° C. (**P<0.01 and ***P<0.001).

FIG. 4M is a graph showing insulin production induced by pUDCA and UDCA from pancreatic β cells.

FIG. 4N is a graph showing IFN-γ production of CD4+ T cells, directly treated with pUDCA, and stimulated with anti-CD3 and anti-CD28.

FIGS. 5A and 5B are graphs showing gastrointestinal (GI) distribution of polymer bile acid (pBA) NPs and dose dependency.

FIG. 5A shows the organ level biodistribution of orally ingested pBAs and control PLGA NPs in GI as compared to free dye. Mice were fasted for 4 h then orally gavaged with DIR-encapsulating pBA NPs (500 mg/kg).

FIG. 5B shows the dose-dependent GI distribution of DIR-loaded pUDCA NPs was also investigated (oral gavage at 50, 100, and 500 mg/kg). The increased fluorescence levels at 4 h post NP ingestion was due to digestive kinetics in the stomach and intestines, and not steady state biodistribution accumulation.

FIGS. 6A-6D are graphs showing accumulation and clearance of pUDCA NPs in pancreas and GI organs.

FIGS. 6A and 6B are graphs of NP uptake in organs measured at time points of 4, 8, 12, and 24 h. Generally, peak uptake of pUDCA in organs at 4 h was found post oral gavage and the particles cleared after 24 h through kidneys.

FIGS. 6C and 6D are graphs of the clearance rates (k) and ΔY (and Tables 4 and 5) from pancreas and small intestine was calculated by non-linear regression curve fitting (one phase decay). Y=(Y0−Plateau)×exp(−k×t)+Plateau, where Y0 is the Y value when t (time) is zero. Plateau is the Y value at infinite times, k is the rate constant. pUDCA showed at least five fold greater pancreatic accumulation compared to controls and its clearance was similar to PLGA.

FIGS. 7A and 7B are graphs showing biodistribution of pUDCA NPs after IV injection. To elucidate the mechanisms of pUDCA NP circulation, the NP were intravenously (IV) injected. pUDCA, PLGA, and the blend NPs (100 mg/kg, 50 μL) were intravenously (IV) administered to B6 mice via tail vein and compare to free dye. Organs and blood were collected and measured at 2 h post ingestion.

FIGS. 8A-8H are graphs showing increased stability in the stomach milieu, permeation through intestinal cells, and pUDCA NP binding to TGR5 with high avidity.

FIG. 8A is a graph of increased permeability through the intestinal barrier modeled using a human epithelial cell line monolayer (Caco-2).

FIG. 8B is a graph of the pancreatic uptake of pUDCA was higher than UDCA.

FIG. 8C is a graph of the rate of endocytosis and exocytosis at 37° C. for pUDCA, a blend of pUDCA and PLGA, and PLGA NPs. A study of valency dependent NP binding to TGR5.

FIG. 8D is a graph of the NPs immobilized on a solid support and FIG. 8E, on the pancreatic β cells.

FIG. 8F is a graph of the subsequent impact on insulin secretion from the pancreatic β cells. To assess the quality of pUDCA multivalency in contrast to particles of the same diameter presenting UDCA repeats (another form of multivalency), PLGA particles were conjugated with different densities of UDCA and used as valency controls. pUDCA's avidity to TGR5 was higher than 2000 monomers of UDCA per PLGA particle (the saturation density). All experiments were performed with 10 samples per group and repeated twice. (*P<0.05, **P<0.01, and ***P<0.001).

FIGS. 9A-9C are graphs showing comparative prevention of T1D.

FIG. 9A shows an experimental scheme. Pancreatic inflammation induced at day 0 with IP injection of cyclophosphamide (CY).

FIG. 9B is a comparative assessment of formulations in prevention. Empty pUDCA (pUDCAEMPTY), monomer UDCA (UDCAEMPTY), pLCA (pLCA_{EMPTY}), and pDCA (pDCAEMPTY) after oral gavage.

FIG. 9C is a graph of the percent diabetic animals (glucose >200 mg/dL).

FIG. 10A is a graph of long-term blood glucose (mg/dL) for saline, soluble insulin, PLGA NPs containing insulin, pUDCA NPS containing insulin, and empty pUDCA NPs.

FIG. 10B is a graph of the GLP-1 secretion as a function of pUDCA (100, 500 mg/kg) compared to UDCA (500 mg/kg).

FIG. 10C, insulin production as a result of TGR5 activation by pUDCA compared to UDCA.

FIG. 11A is a graph of IL-10 (pg/ml) and CCL1 (pg/ml) for pUDCA, UDCA, PLGA, and saline.

FIG. 11B is a graph of the M1/M2 ratio (CD86/CD206) for pUDCA, UDCA, PLGA, and saline.

### DETAILED DESCRIPTION OF THE INVENTION

### I. Definitions

As used herein, the term “nanoparticle” generally refers to a particle having a diameter from about 10 nm up to, but not including, about 1000 nm, preferably from about 60 nm to about 450 nm. The particles can have any shape. Typically, the nanoparticles are spherical and the size is presented as diameter measured in nm as the geometric mean.

As used herein, the term “encapsulated” refers to the agent, for example, a therapeutic and/or an imaging agent, encapsulated within, surrounded by, and/or dispersed throughout a polymeric matrix of the nanoparticle. Alternatively or additionally, the agent can be associated with a polymeric matrix by hydrophobic interactions, charge interactions, van der Waals forces, etc.

As used herein, the term “untargeted” refers to nanoparticles formed of a polymer, such as pUDCA or PLGA, without additional elements, such as targeting moieties, having an increased affinity to a particular cell type or organ. As used herein, the term “targeting moiety” refers to any molecule such as an antibody, ligand, receptor binding moiety, or an active fragment thereof, or an agonist, antagonist, or tissue- or cell-specific targeting molecule, that is used to attach the nanoparticle to a cell in the target organ.

As used herein, the term “active agent” or “biologically active agent” are used interchangeably to refer to a chemical or biological compound that induces a desired pharmacological and/or physiological effect, wherein the effect may be prophylactic, therapeutic and/or diagnostic. The terms also encompass pharmaceutically acceptable, pharmacologically active derivatives of active agents, including, but not limited to, salts, esters, amides, prodrugs, active metabolites, and analogs.

As used herein, the term “excipient”, or “pharmaceutically acceptable excipient”, refers to a pharmacologically inactive substance added to the composition to further facilitate administration of the composition.

As used herein, “oral administration” refers to delivery of the composition to a subject via an oral route. Oral administration can be achieved via oral gavage, or by swallowing of the composition in liquid or solid form. The liquid forms of orally administered compositions can be in a form of a solution, emulsion, suspension, liquid capsule or a gel. Solid forms of orally administered compositions include capsules, tablets, pills, powders, and granules.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve symptoms of, prevent, delay onset of, inhibit progression of, reduce severity of, and/or reduce incidence of the disease, disorder, and/or condition.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a microbial infection may refer to inhibiting survival, growth, and/or spread of the microbe. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, “tolerance” means the reduction in the ability of the immune system to mount an adaptive (T or B-mediated) response to a given antigen.

As used here, “tolerogenic” means the condition or capability of stimulating or increasing tolerance.

As used herein “Treg” includes any T cell that confers suppression. Thus the term encompasses traditional CD4, Foxp3+ Tregs, as well as other CD4 cells that do not express Foxp3 but can be regulatory by secreting IL-10 (Trl cells) among other signals, and CD8 Tregs (Foxp3+ and −) which have also been identified.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

### II. Compositions

The compositions include nanoparticles formed of poly(bile acid) ester polymers. These particles are administered in the absence of therapeutic, prophylactic and/or diagnostic agents incorporated therein or thereon, and, optionally, pharmaceutically acceptable excipients.

Bile acids have been used for decades to enhance oral uptake of drugs. See, for example, Samstein, et al. Biomaterials 29 (2008) 703-708. Bile salts were used to improve the bioavailability of poly(lactide-co-glycolide) (PLGA) nanoparticles by protecting them during their transport through the gastrointestinal tract and enhancing their absorption by the intestinal epithelia. A deoxycholic acid emulsion was shown to protect PLGA nanoparticles from degradation in acidic conditions and enhance their permeability across a model of human epithelium. Oral administration of loaded PLGA nanoparticles to mice, using a deoxycholic acid emulsion, produced sustained levels of the encapsulant in the blood over 24-48 h with a relative bioavailability of 1.81. Encapsulant concentration was highest in the liver, demonstrating targeted delivery to the liver by the oral route.

Studies have now demonstrated that not only does the use of bile acid ester polymers, such as pUDCA, significantly enhance uptake orally, but that the empty particles have antiinflammatory properties. This is believed to be effected through binding of the polymers, e.g., pUDCA, to the TGR5 receptor. With the enhanced surface avidity due to the polymerization and spherical form, empty pUDCA NPs (i.e., not including added therapeutic or prophylactic agent) are effective in reducing inflammation, for example, for treatment of diabetes. Studies show upregulation of GLP-1 through TGR5 binding in the ileum. The anti-inflammatory aspects of UDCA are also magnified in a similar manner.

Based on these findings, the pUDCA NPs are expected and shown to be useful in treating autoimmune and inflammatory diseases and conditions of the pancreas, liver, and colon, including diabetes, pancreatitis, primary biliary cirrhosis (PBC), nonalcoholic steatohepatitis (NASH), IBD, and rCDI (Clostridioides difficile). Generally, the pUDCA NPs provide sustained release of UDCA from pUDCA as the ester bonds deteriorate.

A. Polymers

Generally, the monomers of bile acids suitable for forming poly(bile acid) polymers, are defined by Formula I:

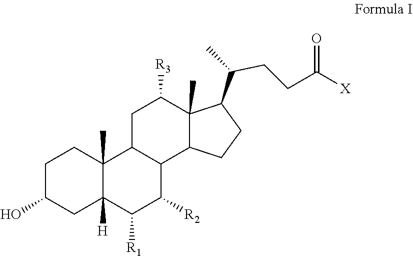

wherein:
R₁, R₂, and R₃ are independently hydrogen or hydroxyl group, and
X is a hydroxyl group at low pH (2-5) that is deprotonated at pH above 5.5. Optionally, X is NHCH₂COOH, NHCH₂COO⁻, NHCH₂CH₂SO₃H, or NHCH₂CH₂SO₃⁻, representing glycine or taurine conjugates (also known as bile salts) of the corresponding bile acid.

The fully protonated hydroxyl group at position X renders the monomers insoluble in water, and the loss of the proton improves the water solubility of the monomers.

The structure of bile acid monomer cholic acid (CA) is shown in Formula II:

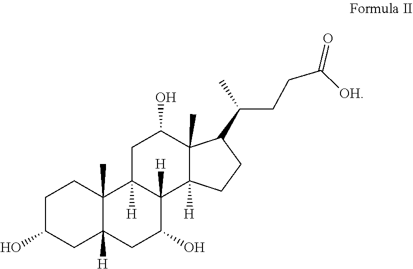

The structure of bile acid monomer lithocholic acid (LCA) is shown in Formula III:

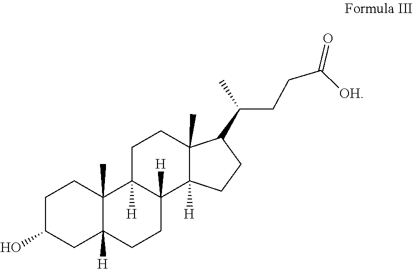

The structure of bile acid monomer deoxycholic acid (DCA) is shown in Formula IV:

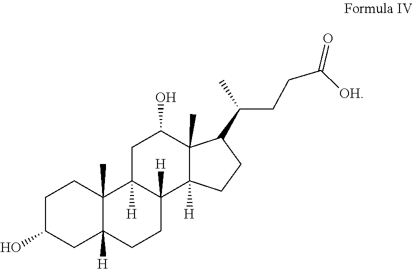

The structure of bile acid monomer cheno-deoxycholic acid (CDCA) is shown in Formula V:

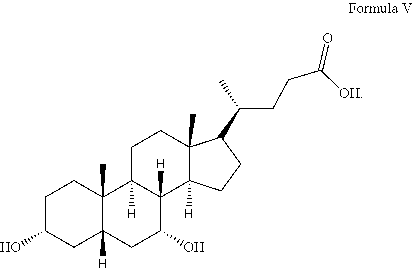

The structure of bile acid monomer urso-deoxycholic acid (UDCA) is shown in Formula VI:

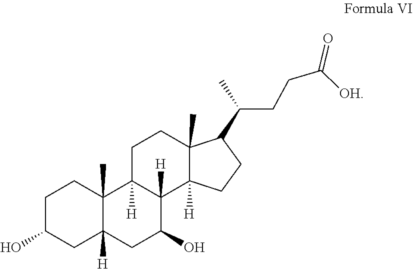

Other suitable bile acids include, but are not limited to, glycocholic acid, taurocholic acid, glycodeoxycholic acid, taurodeoxycholic acid, lithocholic acid, taurolithocholic acid, taurochenodeoxycholic acid, tauroursodeoxycholic acid, glycolithocholic acid, glycochenodeoxycholic acid, glycoursodeoxycholic acid, and taurine conjugates of 3-alpha-7-alpha-12-alpha-22-xi-tetrahydroxy-5-beta-cholestan-26-oic acid (tetrahydroxystero-cholanic acid) and 3-alpha-12 alpha-22-xi-trihydroxy-5-beta-cholestan-26-oic acid.

Other suitable bile acids also include muricholic acids (such as α-muricholic acid, β-muricholic acid, γ-muricholic acid, and ω-muricholic acid), hyodeoxycholic acid, ursocholic acid, isocholic acid, isodeoxycholic acid, isolithocholic acid, isochenodeoxycholic acid, isoursodeoxycholic acid, norcholic acid, nordeoxycholic acid, norlithocholic acid, norchenodeoxycholic acid, norursodeoxycholic acid, apocholic acid, allocholic acid, and their taurine or glycine conjugates.

Additional suitable bile acids are described in Heinken et al., Microbiome 2019, 7:75; Schmidt et al., J Biol Chem, 2010, 285(19):14486-94; Chiang, Compr Physiol, 2013, 3(3): 1191-1212; Sarenac and Mikov, Front Pharmacol, 2018, 9:939; de Haan et al., J Clin Transl Res, 2018, 4(1):1-46; LIPID MAPS Structure Database: Bile acids and derivatives (https://www.lipidmaps.org/data/structure/LMSDSearch.php?Mode=Process ClassSearch&LMID=LMST04).

The above-listed monomers are esterified to produce the poly(bile acid) (PBA) polymers having a molecular weight between about 800 (at least two monomers) and 250,000 Daltons, more preferably between 800 and 50,000 Daltons. In some embodiments, the pUDCA polymers have an Mw value between about 1000 and about 10,000 Daltons, or between about 1200 and about 5,000 Daltons. Room temperature polymerization of bile acids can be carried out using a mixture of diisopropyl carbodiimide (DIC), and a 1:1 salt of dimethyl amino pyridine and p-toluenesulfonic acid (DMAP/PTSA) in mild reaction conditions and without significant cross-linking. Carboiimide activation leads to preferential esterification at carbon 3 and linear polymeric chains. Applied to UDCA, the polymerized UDCA can be defined by Formula VII:

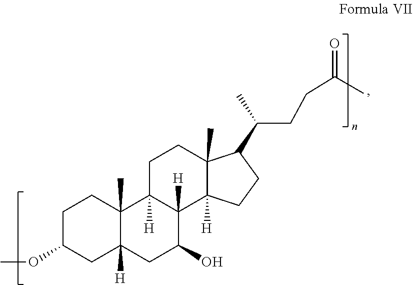

wherein n is a number ranging from between 2-600, preferably between 2 and 100, corresponding to a polymer Mw average in the range of 800-240,000 Daltons.

The degree of branching can vary from a generation 0 (no branches) to higher unlimited number of generations. An exemplary polymerized UDCA with branching is shown in Formula VIII:

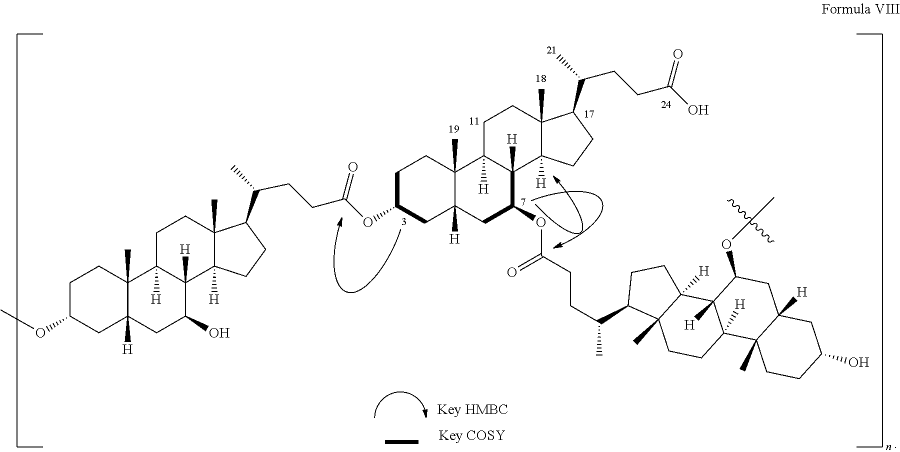

The polymers may be formed from the same monomer, such as UDCA, forming poly(UDCA), or PUDCA. In other embodiments, the polymers may be formed from a mix of bile acid monomers, forming copolymers or monomers coating a polymer bile acid core. In these embodiments, the monomers or polymers may be mixed in any combination, and at any ratio, to form polymeric blends of bile acid ester polymers ranging in molecular weight from between 800 and 250,000 Daltons. Typically, the polymers are linear, but other structures, such as branched, or forked, or dendrimeric, could be used. A dendrimer of poly(bile acids) (dendritic PUDCA, for example), will have a pH stimuli response similar to the linear chain counterparts. This dendritic system will be in a swollen or open state at physiological pH or pH above 6.0. Therefore, it can be easily loaded with drug through non-covalent association with the dendritic polymer or by entrapment in the interstitial cavities formed in the branched system. Low pH will shrink the system, protecting the encapsulant and/or releasing it more slowly. As such, a dendritic bile acid ester polymer may serve as a nanoparticle itself, without the formulation conditions used with linear polymers.

The pUDCA polymers can be formed of ursodeoxycholic acid, glycoursodeoxycholic acid, tauroursodeoxycholic acid, or a combination thereof.

In some embodiments, the monomers or the formed polymeric chains may include moieties with one or more radionuclides, or optical tracers (bioluminescent, chemiluminscent, fluorescent or other high extinction coefficient or high quantum yield optical tracers). Similarly, non-invasive contrast agents such as T1 MR agents in the class of heavy metals (gadolinium, dysprosium, etc.) or T2 contrast agents (iron oxide, manganese oxide, etc.), iodinated agents for X-ray attenuation (CT) and other modalities. The inherent ability of these systems to respond to changes in the pH range of 7 to 2 has significant implications for delivery of therapeutics both to low pH endocytic compartments within cells and/or sites of inflammation characterized by low pH microenvironment or the surrounding environment of tumors. The polymeric chains of these embodiments can be used to form traceable pUDCA nanoparticles, eliminating the need of encapsulating imaging/tracing agents, and enhancing the imaging modalities due to local retention of the imaging agent (confinement of the probe) in the area.

The pUDCA nanoparticles are pH responsive. The polymer backbone shrinks, and the nanoparticles aggregate, in a low pH microenvironment (pH 2-5), and expands at higher pH (pH 6-7.5) to release an encapsulated agent. The pUDCA polymer allows for encapsulation of both hydrophilic and hydrophobic drugs, peptides, proteins, and oligonucleotides. The encapsulated agents are released over time in the higher pH microenvironment of the gut lumen, or generally in organs with pH above 5.5-6.0.

The water solubility of bile acids rises exponentially with increasing pH (Hoffman et al., J. Lipid Res., 33:617-626 (1992)). The polymeric chains of pUDCA and nanoparticles made therefrom aggregate at low pH and become increasingly soluble/dispersed as the pH increases above 5.5. These polymers and nanoparticles are particularly suited for oral drug delivery, as they can protect the agent(s) encapsulated with the nanoparticles from the destructive environment of the stomach. The agent(s) can then be safely released at the neutral pH in the intestines (typically 6-7.4) and target organs, as the polymers begin to dissolve releasing the agent(s).

The nanoparticles can have a mean geometric diameter that is between 50 and 500 nm. In some embodiments, the mean geometric diameter of a population of nanoparticles is about 60 nm, 75 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, or 475 nm. In some embodiments, the mean geometric diameter is between 100-400 nm, 100-300 nm, 100-250 nm, or 100-200 nm. In some embodiments, the mean geometric diameter is between 60-400 nm, 60-350 nm, 60-300 nm, 60-250 nm, or 60-200 nm. In some embodiments, the mean geometric diameter is between 75 and 250 nm. In some embodiments, 30%, 40%, 50%, 60%, 70%, 80%, 90%, or more of the nanoparticles of a population of nanoparticles have a diameter that is between 50 and 500 nm. In a preferred embodiment, the average particle size is 350 nm. Size is measured by conventional techniques, such as optical microscopy.

B. Antiinflammatory Properties of the pUDCA Nanoparticles

While the pUDCA nanoparticles may encapsulate one or more therapeutic, nutritional, diagnostic, and prophylactic compounds in the form of proteins, peptides, carbohydrates, polysaccharides, nucleic acid molecules, organic molecules, or low molecular weight inorganic compounds, the empty nanoparticles have been demonstrated to have intrinsic activity due to the binding to receptors such as the bile acid receptor TGR5 so that the pUDCA polymers function as signaling molecules or metabolic integrators.

The bile acid-activated nuclear receptors such as farnesoid X receptor, pregnane X receptor, constitutive androstane receptor, vitamin D receptor, and G protein-coupled bile acid receptor (e.g., TGR5) play critical roles in the regulation of lipid, glucose, and energy metabolism, inflammation, and drug metabolism and detoxification. TGR5 is a G-protein coupled bile acid receptor known to improve glucose homeostasis by inducing glucagon-like peptide-1 (GLP-1) secretion. Accordingly, the empty pUDCA nanoparticles can exhibit therapeutic and/or prophylactic effects in anti-inflammation (e.g., for treating autoimmune diseases) and/or metabolic regulation (e.g., for controlling blood glucose level and weight).

Polymerization of the bile acid enhances its surface avidity through spatial localization, compared to corresponding nanoparticles formed of the bile acid in its monomeric form. This facilitates the binding of the bile acid to its biological targets, such as bile acid receptors (e.g., TGR5 and farnesol X receptor). The empty pUDCA nanoparticles can release bile acid monomers in a sustained release manner, as the ester bonds connecting the bile acid monomers deteriorate over time.

C. Pharmaceutical Compositions

The nanoparticles can be formulated in liquid or solid form, for oral administration as a single or multiple dosage unit.

The effective dosage may be dependent on the concentration of excipients and how they are added. TGR5 activation results in anti-inflammatory immunity, anti-fibrotic activity, induction and secretion of GLP-1 from enteroendocrine L cells together with increased energy expenditure in adipose tissue 32. pUDCA may not only significantly lower the dose but amplify the range of UDCA function because its monomeric counterpart, UDCA, is an intrinsically weak TGR5 agonist.

The compositions are typically formulated in dosage unit form for ease of administration and uniformity of dosage. It will be understood, however, that the total daily usage of the compositions will be decided by the attending physician within the scope of sound medical judgment. The specific therapeutically effective dose level for any particular subject or organism will depend upon a variety of factors including the disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and other factors well known in the medical arts.

Excipients and/or carriers may be chosen based on the dosage form to be administered, the active agents being delivered, etc. Suitable excipients include surfactants, emulsifiers, emulsion stabilizers, anti-oxidants, emollients, humectants, chelating agents, suspending agents, thickening agents, occlusive agents, preservatives, stabilizing agents, pH modifying agents, solubilizing agents, solvents, flavoring agents, colorants, and other excipients. As used herein, “excipient” does not include any bile acid or polymer thereof.

Suitable emulsifiers include, but are not limited to, straight chain or branched fatty acids, polyoxyethylene sorbitan fatty acid esters, sorbitan fatty acid esters, propylene glycol stearate, glyceryl stearate, polyethylene glycol, fatty alcohols, polymeric ethylene oxide-propylene oxide block copolymers, and combinations thereof.

Suitable surfactants include, but are not limited to, anionic surfactants, non-ionic surfactants, cationic surfactants, and amphoteric surfactants.

Suitable suspending agents include, but are not limited to, alginic acid, bentonite, carbomer, carboxymethylcellulose and salts thereof, colloidal oatmeal, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and combinations thereof.

Suitable antioxidants include, but are not limited to, butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and combinations thereof.

Suitable chelating agents include, but are not limited to, EDTA, and combinations thereof.

Suitable humectants include, but are not limited to, glycerin, butylene glycol, propylene glycol, sorbitol, triacetin, and combinations thereof.

Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

Excipients may include suspending agents such as sterile water, phosphate buffered saline, saline, or a non-aqueous solution such as glycerol.

Particles can be provided as dry powders following spray drying or lyophilization.

Particles may be compressed into tablets, which may in turn be coated with a material such as an EUDRAGIT® to prevent release of the particles after passage through the stomach.

Particles may also be encapsulated in hard or soft gels, such as gelatin and alginate capsules and the enteric formulated soft gels sold by Banner Pharmaceuticals.

Particles may also be formulated for administration to mucosal surfaces, such as the mouth, nasal cavity, oral cavity, pulmonary system, rectal or vaginal surfaces.

Particles may also be provided in a kit, where the material to be delivery is provided separately from the dosage unit, then combined in powder or dry form or in solution prior to use. The agent to be delivered can be entrapped, encapsulated or bound to the bile salt polymers chemically or physically.

### III. Methods of Making Nanoparticles

The pUDCA nanoparticles described herein can be prepared by a variety of methods. The following are representative methods.

A. Solvent Evaporation Microencapsulation

In solvent evaporation microencapsulation, the polymer is typically dissolved in a water immiscible organic solvent and the material to be encapsulated is added to the polymer solution as a suspension or solution in an organic solvent. An emulsion is formed by adding this suspension or solution to a beaker of vigorously stirring water (often containing a surface active agent, for example, polyethylene glycol or polyvinyl alcohol, to stabilize the emulsion). The organic solvent is evaporated while continuing to stir. Evaporation results in precipitation of the polymer, forming solid nanoparticles containing core material.

The polymer or copolymer is dissolved in a miscible mixture of solvent and nonsolvent, at a nonsolvent concentration which is immediately below the concentration which would produce phase separation (i.e., cloud point). The liquid core material is added to the solution while agitating to form an emulsion and disperse the material as droplets. Solvent and nonsolvent are vaporized, with the solvent being vaporized at a faster rate, causing the polymer or copolymer to phase separate and migrate towards the surface of the core material droplets. This phase-separated solution is then transferred into an agitated volume of nonsolvent, causing any remaining dissolved polymer or copolymer to precipitate and extracting any residual solvent from the formed membrane. The result is a nanoparticles composed of polymer or copolymer shell with a core of liquid material.

In solvent removal microencapsulation, the polymer is typically dissolved in an oil miscible organic solvent and the material to be encapsulated is added to the polymer solution as a suspension or solution in organic solvent. Surface active agents can be added to improve the dispersion of the material to be encapsulated. An emulsion is formed by adding this suspension or solution to vigorously stirring oil, in which the oil is a nonsolvent for the polymer and the polymer/solvent solution is immiscible in the oil. The organic solvent is removed by diffusion into the oil phase while continuing to stir. Solvent removal results in precipitation of the polymer, forming solid particles containing core material.

B. Phase Separation Microencapsulation

In phase separation microencapsulation, the material to be encapsulated is dispersed in a polymer solution with stirring. While continually stirring to uniformly suspend the material, a nonsolvent for the polymer is slowly added to the solution to decrease the polymers solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the core material in a droplet with an outer polymer shell.

C. Spontaneous Emulsification

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

D. Coacervation

Encapsulation procedures for various substances using coacervation techniques have been described in the prior art, for example, in GB-B-929 406; GB-B-929 401; U.S. Pat. Nos. 3,266,987; 4,794,000; and 4,460,563. Coacervation is a process involving separation of colloidal solutions into two or more immiscible liquid layers (Dowben, R. General Physiology, Harper & Row, New York, 1969, pp. 142-143). Through the process of coacervation compositions contained of two or more phases and known as coacervates may be produced. The ingredients that contain the two phase coacervate system are present in both phases; however, the colloid rich phase has a greater concentration of the components than the colloid poor phase.

E. Spray Drying

In this method, the polymer is dissolved in organic solvent. A known amount of the active drug is suspended (insoluble drugs) or co-dissolved (soluble drugs) in the polymer solution. The solution or the dispersion is then spray-dried. Typical process parameters for a mini-spray drier (Buchi) are as follows: polymer concentration=0.04 g/mL, inlet temperature=−24° C., outlet temperature=13-15° C., aspirator setting=15, pump setting=10 mL/minute, spray flow=600 Nl/hr, and nozzle diameter=0.5 mm Microparticles ranging between 1-10 microns are obtained with a morphology which depends on the type of polymer used.

F. Fluorine-Mediated Supramolecular Assemblies

Fluorinated bile acid units (either linear or branched) can be synthesized by reaction of a terminal carboxylate or hydroxyl group with an alkylfluorate anhydride (AFAA). The product can extracted into water initiating a fluorophobic effect, in which spontaneous aggregation of the fluorinated building blocks takes place preferentially and differently from a hydrophobic effect. Such assembly is dependent on both the thermal energy, extent of fluorination, enabling some thermodynamic and kinetic control over the final morphology. Fluorophobic-mediated self-assembly will provide the cohesive forces for aggregation and may serve as an intrinsically imageable system through 19F NMR. Fluorinated bile acids will also have a distinctly different biodistribution and clearance time which may serve to enhance the residence time of the system in the GI tract or in the pancreatic regions.

### IV. Methods of Use

A. Routes of Administration

The particles are preferably administered orally, and show enhanced uptake by target organ such as the pancreas, liver, or colon. Oral administration can be achieved via oral gavage, or by swallowing of the composition in liquid or solid form. The liquid forms of orally administered compositions can be in a form of a solution or a liquid gel. Solid forms of orally administered compositions can be in the form of capsules, soft and hard gels, tablets, pills, powders, and granules.

Although described with reference to oral administration, it is understood that the same delivery may be achieved by delivery to a mucosal surface such as the mouth, nasal cavity, lung, lung, rectum or vagina, or delivery through intravenous (i.v.) injection.

The desired dosage may be delivered orally once a day, or multiple times a day. For example, the desired dosage may be delivered orally three times a day, two times a day, once a day, every other day, every third day, every week, every two weeks, every three weeks, or every four weeks. In certain embodiments, the desired dosage may be delivered using multiple daily administrations (e.g., two, three, four, five, six, seven, eight, nine, ten, eleven, twelve, thirteen, fourteen, or more administrations).

B. Disorders to be Treated

A method of preventing, suppressing or treating a disease or condition may include administering to a subject in need thereof an oral dosage unit of the pharmaceutical composition containing the blank pUDCA nanoparticles), optionally to targeted tissue such as pancreas, liver, or colon; prevention, suppression or treatment of one or more symptoms of the disease.

The formulations are useful in treatment of inflammatory diseases and autoimmune and allergenic diseases. The formulations are also efficacious in treating diseases such as diabetes. The empty pUDCA nanoparticles are suitable for treating symptoms or reducing the severity of autoimmune and inflammatory diseases and conditions, such as primary biliary cirrhosis (PBC), nonalcoholic steatohepatitis (NASH), and irritable bowel syndrome (IBS). The empty pUDCA nanoparticles are suitable for controlling blood glucose level, increasing insulin sensitivity and/or treating diabetes, such as Type 1 and Type 2 diabetes. The empty pUDCA nanoparticles are also suitable for helping to control weight and/or treat metabolic disorders. The formulations are also useful in treating Clostridioides difficile infections (such as rCDI).

The empty pUDCA nanoparticles display a higher therapeutic effect or prophylactic effect, compared to empty nanoparticles formed of monomeric bile acid(s) at the same dose (e.g., by weight of the nanoparticles), in which the monomeric bile acid is the same as the repeat unit of the pUDCA polymer in the empty pUDCA nanoparticles.

1. Autoimmune and Inflammatory Diseases

The formulations containing pUDCA nanoparticles, regardless of the presence or absence of any therapeutic or prophylactic agent, can induce a shift of macrophage presentation from M1 to M2, downregulate TNF-α, and/or suppresses expression of pro-inflammatory cytokines.

In some embodiments, the compositions and methods are used to treat chronic and persistent inflammation, which can be a major cause of the pathogenesis and progression of an autoimmune diseases or inflammatory condition. Accordingly, methods of treating inflammatory and autoimmune diseases and disorders can include administering to a subject in need thereof, an effective amount of a particle formulation or a pharmaceutical composition thereof, to reduce or ameliorate one or more symptoms of the disease or condition.

Representative inflammatory or autoimmune diseases and disorders that may be treated include, but are not limited to, nonalcoholic steatohepatitis, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), IgA nephropathy, insulin dependent diabetes (Type 1), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

2. Allergies

A similar methodology can be used to treat allergies, substituting the allergen of interest for the autoimmune stimulus. Typically, particles are administered to a subject in an effective amount to reduce or inhibit an allergy or allergic reaction.

Allergies are abnormal reactions of the immune system that occur in response to otherwise harmless substances. Allergies are among the most common of medical disorders. It is estimated that 60 million Americans, or more than one in every five people, suffer from some form of allergy, with similar proportions throughout much of the rest of the world. Allergy is the single largest reason for school absence and is a major source of lost productivity in the workplace.

An allergy is a type of immune reaction. Normally, the immune system responds to foreign microorganisms or particles by producing specific proteins called antibodies. These antibodies are capable of binding to identifying molecules, or antigens, on the foreign particle. This reaction between antibody and antigen sets off a series of chemical reactions designed to protect the body from infection. Sometimes, this same series of reactions is triggered by harmless, everyday substances such as pollen, dust, and animal danders. When this occurs, an allergy develops against the offending substance (an allergen).

Mast cells, one of the major players in allergic reactions, capture and display a particular type of antibody, called immunoglobulin type E (IgE) that binds to allergens. Inside mast cells are small chemical-filled packets called granules. Granules contain a variety of potent chemicals, including histamine.

Immunologists separate allergic reactions into two main types: immediate hypersensitivity reactions, which are predominantly mast cell-mediated and occur within minutes of contact with allergen; and delayed hypersensitivity reactions, mediated by T cells (a type of white blood cells) and occurring hours to days after exposure.

Inhaled or ingested allergens usually cause immediate hypersensitivity reactions. Allergens bind to IgE antibodies on the surface of mast cells, which spill the contents of their granules out onto neighboring cells, including blood vessels and nerve cells. Histamine binds to the surfaces of these other cells through special proteins called histamine receptors. Interaction of histamine with receptors on blood vessels causes increased leakiness, leading to the fluid collection, swelling and increased redness. Histamine also stimulates pain receptors, making tissue more sensitive and irritable. Symptoms last from one to several hours following contact. In the upper airways and eyes, immediate hyper-sensitivity reactions cause the runny nose and itchy, bloodshot eyes typical of allergic rhinitis. In the gastrointestinal tract, these reactions lead to swelling and irritation of the intestinal lining, which causes the cramping and diarrhea typical of food allergy. Allergens that enter the circulation may cause hives, angioedema, anaphylaxis, or atopic dermatitis.

Allergens on the skin usually cause delayed hypersensitivity reaction. Roving T cells contact the allergen, setting in motion a more prolonged immune response. This type of allergic response may develop over several days following contact with the allergen, and symptoms may persist for a week or more.

Allergens enter the body through four main routes: the airways, the skin, the gastrointestinal tract, and the circulatory system. Airborne allergens cause the sneezing, runny nose, and itchy, bloodshot eyes of hay fever (allergic rhinitis). Airborne allergens can also affect the lining of the lungs, causing asthma, or conjunctivitis (pink eye). Exposure to cockroach allergens has been associated with the development of asthma. Airborne allergens from household pets are another common source of environmental exposure. Allergens in food can cause itching and swelling of the lips and throat, cramps, and diarrhea. When absorbed into the bloodstream, they may cause hives (urticaria) or more severe reactions involving recurrent, non-inflammatory swelling of the skin, mucous membranes, organs, and brain (angioedema). Some food allergens may cause anaphylaxis, a potentially life-threatening condition marked by tissue swelling, airway constriction, and drop in blood pressure. Allergies to foods such as cow's milk, eggs, nuts, fish, and legumes (peanuts and soybeans) are common. Allergies to fruits and vegetables may also occur. In contact with the skin, allergens can cause reddening, itching, and blistering, called contact dermatitis. Skin reactions can also occur from allergens introduced through the airways or gastrointestinal tract. This type of reaction is known as atopic dermatitis. Dermatitis may arise from an allergic Dermatitis may arise from an allergic response (such as from poison ivy), or exposure to an irritant causing nonimmune damage to skin cells (such as soap, cold, and chemical agents). Injection of allergens, from insect bites and stings or drug administration, can introduce allergens directly into the circulation, where they may cause system-wide responses (including anaphylaxis), as well as the local ones of swelling and irritation at the injection site.

3. Diabetes

Diabetes, or diabetes mellitus, is due to either the pancreas not producing enough insulin or the cells of the body not responding properly to the insulin produced. There are three main types of diabetes mellitus:

Type 1 diabetes results from the pancreas' failure to produce enough insulin or active insulin; this form was previously referred to as “insulin-dependent diabetes mellitus” (IDDM) or “juvenile diabetes”;

Type 2 diabetes begins with insulin resistance, a condition in which cells fail to respond to insulin properly. As the disease progresses a lack of insulin may also develop; this form was previously referred to as “non insulin-dependent diabetes mellitus” (NIDDM) or “adult-onset diabetes”; and

Gestational diabetes, the third main form, occurs when pregnant women, without a previous history of diabetes, develop a high blood sugar level.

Type 1 diabetes must be managed with insulin injections. Type 2 diabetes may be treated with medications with or without insulin. Gestational diabetes usually resolves after the birth of the baby.

People with Type 1 diabetes need insulin therapy to survive. Many people with Type 2 diabetes or gestational diabetes also need insulin therapy. Medications used for treating T2D include over 20 types of injectable insulin, and orally administered drugs such as meglitinides, sulfonylureas, metformin, canagliflozin, dapagliflozin, thiazolidinediones, pioglitazone, rosiglitazone, acarbose, pramlintide, exenatide, liraglutide, long-acting exenatide, albiglutide, dulaglutide, and dipeptidyl peptidase-4 (DPP-IV) inhibitors (sitagliptin, saxagliptin, linagliptin). These agents are collectively referred to as “anti-diabetics”.

The compositions can be used to treat the inflammation of the pancreas (pancreatitis), the liver (hepatitis), or the colon (IBD). The pUDCA nanoparticles encapsulating a therapeutic and/or imaging agent, can pass through the fenestrated vasculature of an inflamed tissue, and are retained longer within the inflamed tissue, due to their size, compared to biologics or small molecule drugs (1-10 nm). They are also effectively internalized by antigen-presenting cells (such as macrophages and dendritic cells).

Two forms of pancreatitis, acute and chronic pancreatitis, can be treated with oral administration of the pUDCA compositions.

Acute pancreatitis is a sudden inflammation that lasts for a short time.

It may range from mild discomfort to a severe, life-threatening illness. In severe cases, acute pancreatitis can result in bleeding into the gland, serious tissue damage, infection, and cyst formation. Severe pancreatitis can also harm other vital organs such as the heart, lungs, and kidneys.

Chronic pancreatitis is long-lasting inflammation of the pancreas. It most often happens after an episode of acute pancreatitis. Heavy alcohol drinking is another big cause. Damage to the pancreas from heavy alcohol use may not cause symptoms for many years, but then the subject may suddenly develop severe pancreatitis symptoms. Subjects with acute pancreatitis are treated with IV fluids and pain medications in the hospital. Chronic pancreatitis can be difficult to treat. It involves pain relief and improved nutrition. Subjects are generally given pancreatic enzymes or insulin.

The inflammation of the liver (hepatitis) is characterized by the presence of inflammatory cells in the tissue of the organ. Hepatitis may occur with limited or no symptoms, but often leads to jaundice (a yellow discoloration of the skin, mucous membrane, and conjunctiva), poor appetite, and malaise. Hepatitis is acute when it lasts less than six months and chronic when it persists longer.

Acute hepatitis can be self-limiting (healing on its own), can progress to chronic hepatitis, or, rarely, can cause acute liver failure. Chronic hepatitis may have no symptoms, or may progress over time to fibrosis (scarring of the liver) and cirrhosis (chronic liver failure). Cirrhosis of the liver increases the risk of developing hepatocellular carcinoma.

Viral hepatitis is the most common cause of liver inflammation. Other causes include autoimmune diseases and ingestion of toxic substances (notably alcohol), certain medications (such as paracetamol), some industrial organic solvents, and plants. Antiretroviral drugs such as tenofovir and entecavir are used for the treatment of chronic hepatitis B.

4. Inflammatory Bowel Disease

Inflammatory bowel disease (IBD) is a broad term that describes conditions with chronic or recurring immune response and inflammation of the gastrointestinal tract. The two most common inflammatory bowel diseases are ulcerative colitis and Crohn's disease. Inflammation affects the entire digestive tract in Crohn's disease and only the large intestine in ulcerative colitis. Both illnesses are characterized by an abnormal response to the body's immune system.

Crohn's disease is treated with medications designed to suppress the immune system's abnormal inflammatory response that causes the symptoms. Suppressing inflammation offers relief from common symptoms like fever, diarrhea, and pain, and healing of the intestinal tissues. Combination therapy could include the addition of a biologic to an immunomodulator. As with all therapies, there are risks and benefits of combination therapies. Combining medications with immunomodulatory therapies can increase the effectiveness of IBD treatment.

5. Metabolic Disorders and Weight Control

The formulations containing pUDCA nanoparticles, regardless of the presence or absence of any therapeutic or prophylactic agent, can upregulate GLP-1 in alpha cells, enhance insulin production in beta cells, and/or upregulate GLP-1 in L cells (ileum), causing a cascade effect on glucose metabolism in the liver as GLP-1 flows from pancreas to liver in portal vein. The formulations can also cause upregulation of AMP-activated protein kinase (AMPK), an enzyme that plays a role in cellular energy homeostasis, largely to activate glucose and fatty acid uptake and oxidation. The formulations can also improve the function of mitochondria, such as increasing mitochondrial membrane potential and respiration (oxygen consumption, ATP production, etc.).

The formulations containing pUDCA nanoparticles, regardless of the presence or absence of any therapeutic or prophylactic agent, can also induce a shift of macrophage presentation from M1 to M2 in beta islet infiltrating macrophages and exert systemic effects in reducing adipose inflammation and insulin resistance.

Accordingly, the formulations containing pUDCA nanoparticles can be used to treat metabolic disorders, control blood glucose level, control blood lipid level, and/or control weight. Exemplary metabolic disorders include glucose metabolism disorders, lipid metabolism disorders, metabolic syndrome X, and mitochondrial diseases.

6. CNS-Related Disorders

The formulations containing pUDCA nanoparticles, regardless of the presence or absence of any therapeutic or prophylactic agent, can be used to treat or prevent CNS-related disorders, especially neurodegenerative diseases such as Alzheimer's disease and Parkinson's disease.

UDCA can reduce IL-1β and nitric oxide, and downregulate TNF-α in rat microglia (Joo et al., Archives of Pharmacal Research, 2003, 26(12):1067-1073; Joo et al., Archives of Pharmacal Research, 2004, 27:954). UDCA can also improve mitochondrial function and redistributes Drp1 in fibroblasts from patients with either sporadic or familial Alzheimer's disease (Bell et al., J Mol Biol, 2018, 430(21):3942-3953).

The present invention will be further understood by reference to the following non-limiting examples.

The Examples show that pUDCA works through parallel more than additive mechanisms involving protective transport, enhancement in recognition, metabolic and anti-inflammatory immune signals. The formulation of pUDCA NP begins with the monomer UDCA, well known for its established medicinal benefits, followed by polymerization and then formulation into NP. The polymerization and formulation steps expand the benefits beyond what can be achieved with monomer alone or even monomer on the surface of particles, as validated in the mechanistic studies that follow. The efficacy of pUDCA is due to multiple mechanisms. The first is protective transport facilitating improvements in pharmacokinetics and biodistribution of encapsulated agents.

The present invention will be further understood by reference to the following non-limiting examples.

### EXAMPLES

### Example 1: Polymeric BAs not Only Facilitate the Formulation of Orally Ingestible Therapeutic Nanoparticles but Also Provide a Broad-Spectrum of Bioactivity

There are two reasons the nanoparticle provide a broad-spectrum of activity:
1) they can be protective in nature, and increase intestinal permeation and thus the systemic bioavailability of associated agents; and
2) they possess signaling functions that can regulate glucose metabolism and immunity through binding of BA receptors and thus function as effector therapeutic systems.

The rationale for polymerization was based on the notions that: 1) polymerization facilitates a strategy for encapsulation and release of a wide range of therapeutics of interest including insulin. In other words, solid, stable, biodegradable polymeric carriers in contrast to monomeric BA micelles, which are inherently unstable. 2) Fabrication of such polymeric NPs enable sustained release of encapsulated agents if the polymers are degradable in aqueous environments. 3) Polymeric BA systems as robust carriers present BA differently (in close proximity and higher density) than, say, BA monomers hybridized on the surface of another type of polymer NPs. Furthermore, if the BA monomer has intrinsic therapeutic effect, then this effector function is be amplified with polymerization and its bioavailability is longer lasting in contrast to BA monomers on particles which may be easily released from the surface after oral ingestion. Furthermore, the sustained availability of BA over the drug release period may be a desired element for combinatorial more than additive activity. 4) The pH stimulus response of BAs which is due to their ionization potential and protonation at low pH offers stomach protection while enhancing BA with multivalency for binding to its receptors. This multivalent response not only amplifies the degree of ionization, but also kinetically amplifies the low pH protection and higher pH deprotection response time as particles transit from the stomach to the intestinal milieu. 5) Polymeric multivalency results in high binding avidity to BA receptors which results in conversion of a weak BA agonist into a stronger form upon polymerization. Stronger agonists enable greater receptor activation and therapeutic signaling functions at lower doses.

UDCA has an established record of use for lowering insulin resistance in Type 2 Diabetes (T2D), however this usage is dose-dense (typically 40-450 mg/kg in mice and for 2-20 weeks orally). UDCA is rarely tested in T1D since it mainly impacts insulin sensitivity. The functional impact of pUDCA extends beyond improvements in transport of encapsulated agent (such as insulin) in addition to amplification of its effector function beyond what the monomer can achieve on its own. UDCA can trigger protein kinase cascade cell activation, regulate glucose, and energy homeostasis if it stably binds to extracellular Takeda G-protein coupled receptors (TGR5), and can regulate nuclear factor κB (NF-κB) and signal kinases such as protein kinase B (Akt). TGR5 activation also results in anti-inflammatory immunity, anti-fibrotic activity, induction and secretion of GLP-1 from enteroendocrine L cells together with increased energy expenditure in adipose tissue. pUDCA may not only significantly lower the dose but amplify the range of UDCA function because its monomeric counterpart, UDCA, is an intrinsically weak TGR5 agonist.

From the standpoint of improvements in insulin transport, biodistribution and pharmacokinetics, BAs are natural emulsifiers. Thus, biodegradable, polymeric BA would be even better in solubilization of lipids and fats in the body. Generally, BAs function as digestive aid through their self-assembly with lipids into micelles; enabling better molecular biodistribution and blood circulation of orally ingested fatty substances. Bile and pancreatic digestive juices are known to secrete into the duodenum and bile, specifically, is shuttled from the ileum back to the liver through portal circulation then once again returned back to the intestines for further digestion of ingested fatty foods. This circulatory action of BAs from the intestines to the bile duct and back is a process termed, “Enterohepatic Circulation”. Because of the enhanced binding of polymeric BA NP, biodistribution is affected and circulation lifetime is longer.

Methods and Materials

The methods used in the Examples were as follows.

Reagents and Antibodies.

All bile acids, para-toluenesulfonic acid, 4dimethylaminopyridine (DMAP), poly(vinyl alcohol) (PVA), Tween 20, pepsin, triamterene, lipopolysaccharide (LPS), and ovalbumin (OVA) were obtained from Sigma and Sigma-Aldrich. Cyclophosphamide (CY), anhydrous methylene chloride, anhydrous pyridine, diisopropyl carbodiimide, and anhydrous methanol were purchased from ACROS. Poly(lactic-co-glycolic acid) (PLGA, inherent viscosity 0.55-0.75 dL/g, carboxyl terminal) from Durect was used as a control polymer. Rapamycin (RAPA, LC Laboratories), mouse insulin (INS, R&D systems), 1,1′-dioctadecyl-3,3,3′,3′-tetramethylindotricarbocyanine iodide (DIR, Biocompare), and coumarin 6 (ACROS) were encapsulated in NPs. EUDRAGIT® FS 30D was obtained from Evonik and CpG was purchased from InvivoGen. Antibodies for CD8 (APC), CD44 (PE), CD4 (APC), CD25 (Alexa Fluor-700), CD11c (PE-Cy7), F4/80 (Alexa Fluor-647), F4/80 (Alexa Fluor-700), and CD206 (FITC) were obtained from BioLegend. Foxp3 (PE) and CD86 were purchased from Invitrogen and eBioscience, respectively. Recombinant Human GPCR TGR5 protein, Atto565-conjugated TGR5 antibody, and blocking buffer were obtained from Abcam and used for competitive binding study.

Cells.

The human colon adenocarcinoma Caco-2 cells were purchased from ATCC. The cells were cultured in Dulbecco's Modified Eagle's Medium (DMEM, Life Technologies) containing 4.5 g/L glucose, 10% Fetal Bovine Serum (FBS, Atlanta Biologicals), antibiotics (100 units/mL penicillin and 100 μg/mL streptomycin, Gibco), and 1% nonessential amino acid (NEAA, Gibco). Long bones and spleens were harvested from mice (C57BL/6 or Rag2/OTII) post cervical dislocation. Bone marrow was eluted from long bones and spleens were macerated using Roswell Park Memorial Institute (RPMI)-1640 (Life Technologies) media supplemented with 10% FBS. Red blood cells (RBCs) in the sample were lysed using ammonium-chloride-potassium (ACK) lysing buffer (Lonza). Bone-marrow derived macrophages (BMMs) were cultured in Roswell Park Memorial Institute (RPMI, Life Technologies) media with macrophage colony-stimulating factor (MCSF, 10 ng/mL, Sigma-Aldrich). Bone-marrow derived dendritic cells (BMDCs) were generated using a conventional expansion protocol in which 5×10⁵ cells/mL were plated in RPMI supplemented with 20 ng/mL GM-CSF (Sigma-Aldrich) and cultured for 5 days. On day 5, non-adherent cells were collected and cultured in GM-CSF media for an additional 2 days. CD4+ T cells were purified from splenocyte population in C57BL/6 using an EasySep™ Mouse CD4+ T Cell Isolation Kit (STEMCELL Technologies). All cells were cultured at 37° C. in a humidified atmosphere of 5% C₀2.

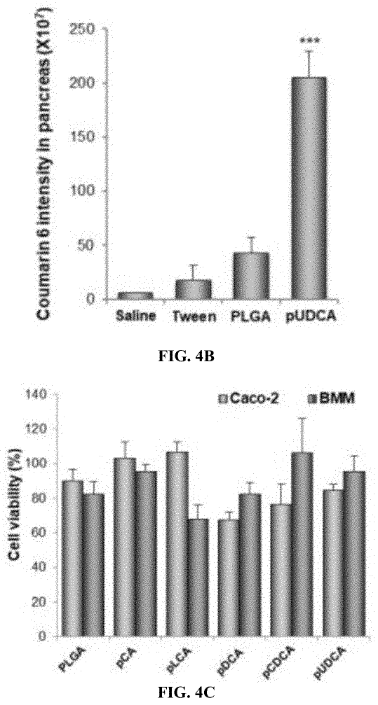

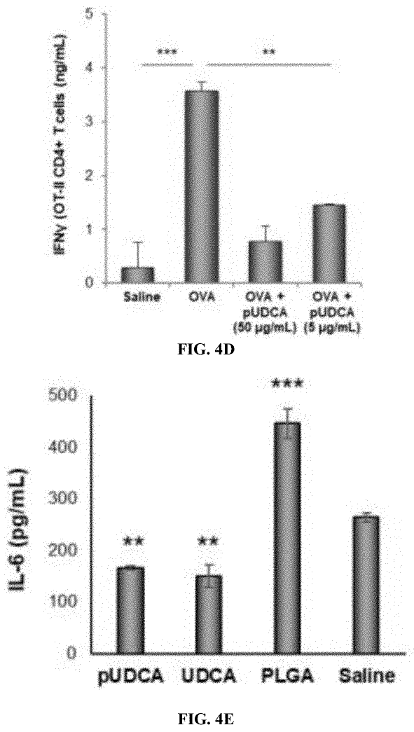

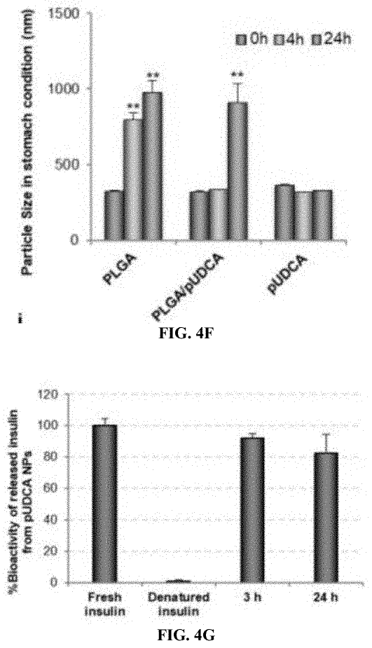

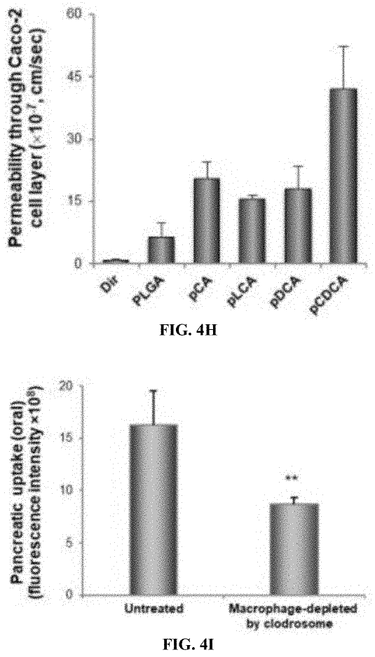

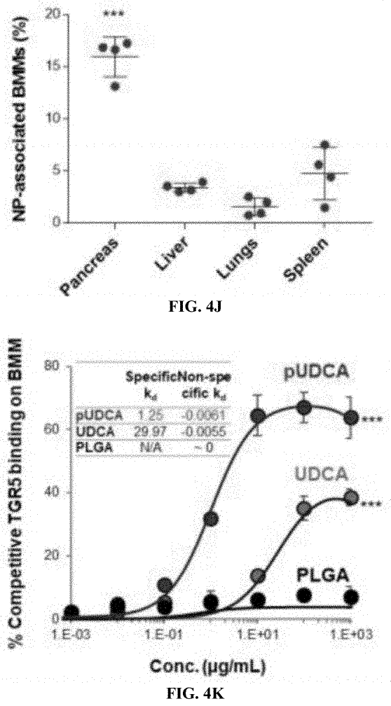

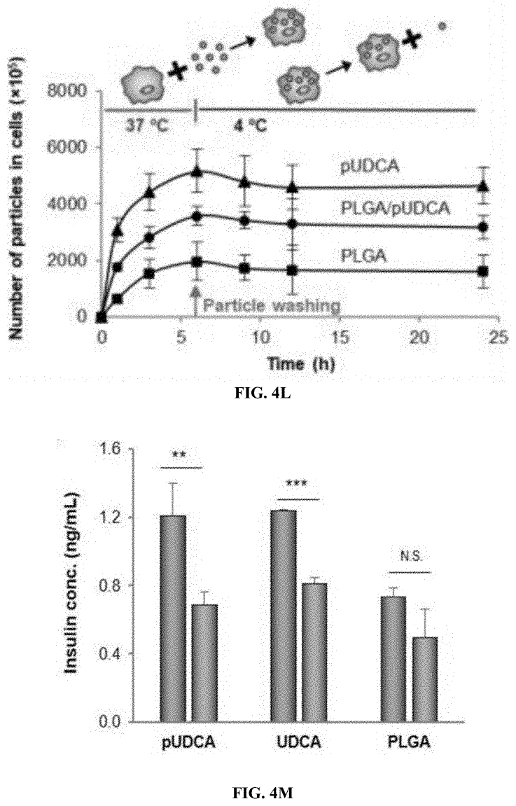

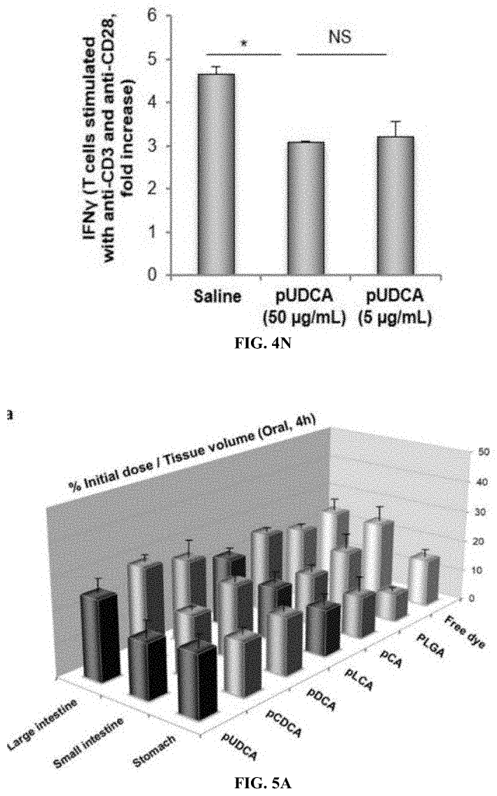

For testing insulin production from pancreatic β cells promoted by activation of TGR5 receptor, the mouse pancreatic β cell line (MIN6, ATCC) cells were incubated in Hank's balanced salt solution (HBSS, Life Technologies) containing 3 mM glucose for 2 h and then for 30 mM in HBSS with 25 mM glucose and UDCA, PLGA or pUDCA NPs (40 μg/mL). Concentration of insulin was measured using an Ultrasensitive Insulin ELISA kit (ALPCO). The same experiment was performed in the presence of TGR5 antagonist, triamterene (50 μg/mL) as a control to differentiate inherent insulin production from the cells without TGR5 activation and used to normalize the results. Bioactivity of released insulin from pUDCA were measured using Chinese hamster ovary cells that were transfected with the gene to express insulin receptor (CHO INSR cells, ATCC). The released insulin from pUDCA at 3 or 24 h was incubated with CHO INSR cells for 1 h and phosphorylated protein kinase B (pAkt) level was measured by ELISA (Abcam). The pAkt production from CHO cells incubated with fresh or denatured insulin was compared to calculate percent bioactivity. See FIG. 4G.

Animals.

C57BL/6 mice (B6, 6-8-week-old, female) were obtained from Harlan Sprague Dawley Inc. NOD mice (NOD/ShiLtJ, 8-week-old, female) and Nude mice (athymic nude, nu/nu, 7-week-old, female) were supplied by Jackson Laboratory. The mice were housed in autoclaved micro-isolator cages that were placed in a positive pressure containment rack. Ossabaw Swine (17-month-old, 42 kg) derived from the Ossabaw barrier islands were used. All experiments and maintenance were carried out according to an approved protocol from the Yale University Institutional

Animal Care and Use Committee.

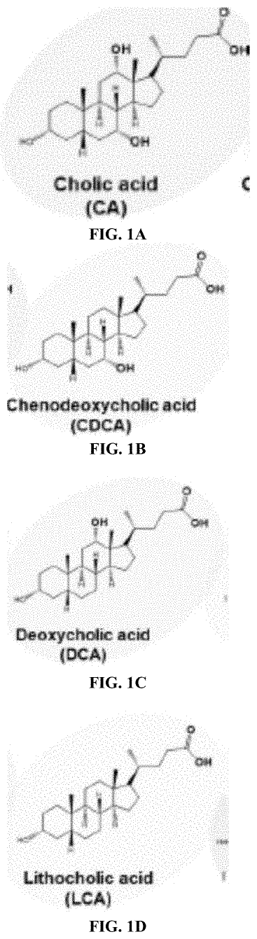

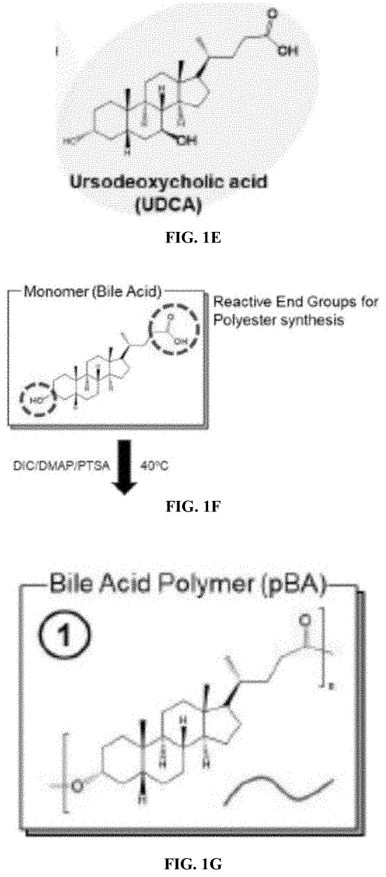

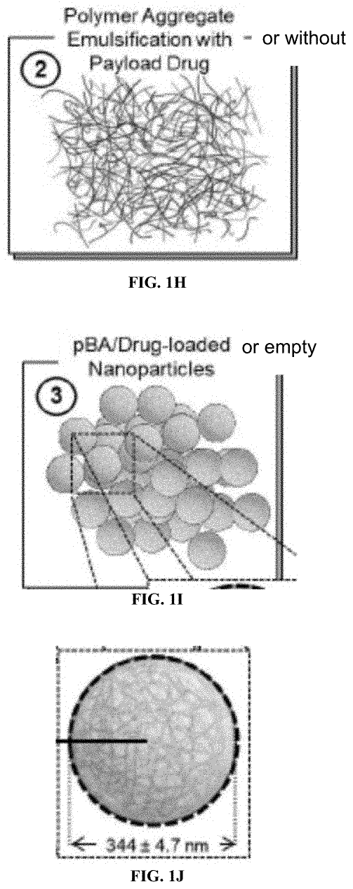

Polymer Synthesis and Nanoparticle (NP) formulation. Poly(bile acid)s (pBAs) were synthesized by esterification of the carbon 24 group (FIGS. 1F-1J). BAs (5.4 mmol), paratoluenesulfonic acid (0.652 mmol), and DMAP (0.652 mmol) were added in 60 mL of a 5:1 anhydrous methylene chloride to anhydrous pyridine solvent mixture and stirred at 40° C. to yield a clear solution. To the reaction mixture, 6.92 mmol of diisopropyl carbodiimide was added and the reaction was allowed to proceed for 2 h in the nitrogen atmosphere. The polyester product, pBA, was precipitated into 400 mL of cold anhydrous methanol collected by centrifugation (Centrifuge 5810R, Eppendorf) and dried to retain a white powder. Polymerization was confirmed by nuclear magnetic resonance (NMR) and gel permeation chromatography (GPC). ¹H and 2D- (COSY, DQF-COSY, HSQC and HMBC) NMR spectral data for UDCA and poly(ursodeoxycholic acid) (pUDCA) were recorded on an Agilent NMR spectrometer (Agilent) at 600 MHz with a 3 mm cold probe or 400 MHz and ¹³C NMR data was measured at 100 MHz magnetic field. Chloroform-d₁ (99.96%, Cambridge Isotope Laboratories, Inc.) was used as the deuterated NMR solvent and solvent reference signals (δ_{H} 7.25, δ_{C} 76.98) for all of the NMR experiments. The molecular weight (MW) for pBAs (10 mg/mL in chloroform) were evaluated with GPC using a Waters HPLC system equipped with a model 1515 isocratic pump, a 717 plus autosampler, and a 2414 refractive index (RI) detector with Waters Styragel columns HT6E and HT2 in series. Chloroform was utilized as the mobile phase with a flow rate of 1 mL/min and both the columns and RI detector were maintained at 40° C. MW characteristics were determined relative to a calibration curve generated from narrow polydispersity polystyrene standards (Aldrich Chemical). Empower II GPC software was used for running the GPC instrument and subsequent chromatographic analysis. pBA or PLGA or the mixture (50/50, w/w) NPs encapsulating dyes (DIR or coumarin 6), drugs (RAPA or INS) or iron oxide (synthesized as previously described⁷²) were formulated using an water-in oil-in water (W/O/W) double emulsion technique (FIGS. 1F-1J). Polymers or the mixture (100 mg) was dissolved in 2 mL chloroform containing DIR (1 mg), coumarin 6 (10 mg), RAPA (10 mg) or iron oxide (1 mg). Phosphate-buffered saline (PBS, 100 μL) or the PBS containing INS (10 μg) was added dropwise to the chloroform polymer solution while vortexing and homogenized using an IKA T25 Digital Ultra-Turrax (IKA). This dispersant phase was then added dropwise to a continuous phase of 5% PVA and homogenized. The mixture was then added dropwise to 200 mL of 0.2% PVA and left stirring for 2 h to evaporate the solvent. NPs were collected by centrifugation at 12,000 rpm for 20 min at 4° C. and then washed 3 times with deionized water. The particles were lyophilized and stored at −20° C. The hydrodynamic diameter and surface charge of NPs were measured by a Malvern Zetasizer. A dispersion of NPs was filtrated through a 0.45 μm Millipore filter into cuvettes prior to the measurements. Dynamic light scattering was measured by back-scattering at a detection angle of 173° at the wavelength of 532 nm and the hydrodynamic radius was calculated using the Stokes-Einstein equation. The morphology of the NPs was observed by Hitachi S-4800 High Resolution scanning electron microscopy (SEM, Norcross). A dispersion of NPs in ethanol (2 μL) was placed on the wafer substrate and dried at room temperature. The sample was mounted on the aluminum sample holder and then gold sputtered. The NPs were observed with an accelerating voltage of 15 kV at a working distance of 4 mm Release of DIR and insulin was measured in the stomach-mimicking media. NPs were dispersed in the media (citrate buffer solution, pH 2.0) at 37° C. in the presence of pepsin (10 mg/mL). At each time point, NPs were centrifuged, and supernatant was collected to measure the amount of DIR released from the particles using a plate reader (λₑₓ 750 nm, λₑₘ 790 nm, SpectraMax M5, Molecular Devices). Insulin release was quantified by the BCA assay. EUDRAGIT® coated PLGA (PLGA@EUDRAGIT®) was prepared by dispersing PLGA in 5 wt % EUDRAGIT® solution and centrifugation.

Permeability of NPs Though Human Intestinal Epithelial Cell Layer.

Caco-2 cells seeded at 7×10⁴ cells/cm² on 0.4 μm pore transwell filters (Corning). Cells were grown to confluence and allowed to mature for approximately 30 days at 37° C. and 5% CO₂. Prior to performing permeability studies, the transepithelial electrical resistance (TEER) was measured using an epithelial voltohmmeter (EVOM™ Epithelial Volt/Ohm Meter, World Precision Instruments, Inc.). Confluent cell layers with TEER values greater than 300 Ω·cm² were used for permeability and cytotoxicity studies. A dispersion of 1 mg/mL DIR-loaded NPs or a solution containing an equivalent concentration of soluble DIR was prepared in phenol-free HBSS (Life Technologies) containing 25 mM glucose and added to the apical chamber of the transwell filter. HBSS in the basolateral chamber was sampled and replaced with fresh media at each time point. The rate of cumulative DIR transport to the basolateral chamber gave the flux, dQ/dt. The apparent permeability (Pₐₚₚ) was calculated by (1).

$${P}_{app} = \frac{dQ / dt}{{C}_{0} \times A} \qquad Eq , 1$$

where C₀ is the initial concentration of total DIR in the apical chamber and A is the area of the transwell filter.

TGR5 Binding Studies.

The competitive binding of pUDCA, UDCA, and PLGA NPs to macrophages saturated with an Atto565-conjugated TGR5 antibody was performed. The cells (10⁵ cells/well) in 96 well plates were incubated with an access amount (4 μg/mL) of the fluorescently labeled TGR5 antibody at 4° C. for 2 h and subsequently exposed to different concentration of NPs. At 2 h post incubation, the cells were washed three times with PBS and the number of Atto565-TGR5 antibodies bound on the cells were measured using a plate reader. The specific and non-specific k_{d} were calculated by non-linear fitting using a site saturation total binding equation, Y=Bₘₐₓ×X/(k_{d}+X)+NS×X, where Bₘₐₓ is the maximum specific binding, k_{d} is the equilibrium dissociation constant, and NS is the slope of nonspecific binding. To study valency dependent NP binding to TGR5 on pancreatic β cells (10⁶ cells/well), UDCA monomer was biotinylated and conjugated onto avidinated PLGA NP surface. PLGA (100 mg) in 2 mL chloroform was added dropwise to a mixture of avidin-palmitate in PBS (10 mg/2 mL) and 5% PVA 2 mL, and homogenized. UDCA was conjugated with biotin (1:1 molar ratio) using the EDC/NHS chemistry prior to immobilization of biotinylated UDCA (0, 50, 250, and 1000 ng/mL) to avidinated PLGA NPs (5 mg/mL). To prepare plated TGR5, recombinant TGR5 receptor (5 μg/mL) was coated on the plate overnight and the non-specific binding sites were blocked using a protein blocking buffer. The TGR 5 receptors on the plates were incubated with an access amount (4 μg/mL) of the fluorescently labeled TGR5 antibody at 4° C. for 2 h and subsequently exposed to different concentration of NPs. At 2 h post incubation, the plates were washed three times with PBS and the number of Atto565-TGR5 antibodies bound on the receptors were measured using a plate reader.

Data was fit to the following competitive inhibition equation, (using Graphpad Prism), which gave an estimate of pUDCA at which 50% of the labeled antibody is competed off (EC₅₀) and its affinity constant (Kᵢ):

$$\begin{matrix}F = {F}_{Initial} + [ \frac{( {F}_{Final} - {F}_{Initial} )}{( 1 + {10}^{[ pUDCA ] - Log ( {EC}_{50} )} )} ] & Eq . 2 \\ Log ( {EC}_{50} ) = Log [ {10}^{log {K}_{i} * ( 1 + \frac{{C}_{Anti}}{{K}_{D , Anti}} )} ] & Eq . 3\end{matrix}$$

where
F=Fluorescence change with competition against labeled TGR5 antibody;
[pUDCA]=Concentration of pUDCA;
Fᵢₙᵢₜᵢₐₗ=Upper plateau of fluorescence or initial fluorescence;
F_{Final}=Lower plateau of fluorescence or final fluorescence;
EC₅₀=Concentration of pUDCA that lowers the total fluorescence by 50%;
Kᵢ=pUDCA affinity constant;
C_{Anti}=Concentration of labeled anti-TGR5 antibody;
K_{D,Anti}=Affinity constant of the labeled anti-TGR5 antibody to TGR5 receptors estimated in the nanomolar range.

It was estimated that a shell of thickness equivalent to the molecular diameter of an UDCA can incorporate approximately 2000 UDCA monomers on a 350 nm diameter pUDCA particle.

Quantitation of cellular endocytosis and exocytosis rates of NPs.

BMMs were seeded in a 96 well plate (10⁵ cells/well) and DIR-loaded pUDCA, PLGA, and PLGA/pUDCA blend NPs (100 μg/mL) were added to the media. Cells were incubated for 1, 3, and 6 h at 37° C., and endocytosis of NPs was measured using a plate reader. After washing the cells and replacing with new media, exocytosis of NPs was monitored at 37° C. or 4° C. by measuring released DIR-labeled NPs from BMMs to media over time. The equilibrium endocytosis—exocytosis reaction can be simplified to:

$$[ P ] + [ C ] {⇌}_{ {k}_{exo}}^{ {k}_{endo }} [ PC ] \qquad Eq . 4$$

where
[P]=concentration of particles in the media (number of particles/mL)
[C]=concentration of cells in the media (number of cells/mL)
[PC]=concentration of particles associated with cells (number of particle-cell/mL)
kₑₓₒ=rate of exocytosis (t⁻¹)
k_{endo}=rate of endocytosis (([P]·t⁻¹)
   then,

$$\frac{d [ PC ]}{dt} = {k}_{endo} [ P ] [ C ] - {k}_{exo} [ PC ] \qquad Eq . 5$$

In terms of a signal reporting on the endocytosis and exocytosis process, which is a fluorescence signal associated with each process [S].

$$\frac{dS}{dt} = {k}_{endo} [ P ] S - ( {k}_{endo} [ P ] + {k}_{exo} ) S \qquad Eq . 6$$

This differential equation has a solution between the two extreme limits of no uptake to maximal uptake:

$$S = \frac{{k}_{endo} [ P ] {S}_{max} [ 1 - {e}^{- ( {k}_{endo} [ P ] + {k}_{exo} ) t} ]}{{k}_{endo} [ P ] + {k}_{exo}} + {S}_{0} \qquad Eq . 7$$

S₀=signal at an arbitrary start time t₀
This analysis and fitting for kendo and kexo are done using two plots.

### Exocytosis Phase

$$\frac{dS}{dt} = - {k}_{exo} S so Ln ( \frac{{S}_{0}}{{S}_{t}} ) = {k}_{exo} t \qquad Eq . 8$$

where St is the signal at any time (t)
S0 is the signal at an arbitrary time (t₀)

### Association Phase

The association phase is analyzed in terms of two plots:
dS/dt against S gives

$$Slope = - ( {k}_{endo} [ P ] + {k}_{exo} ) , \\ Intercept = {k}_{endo} [ P ] {S}_{max} at \frac{dS}{dt} = 0 \qquad Eq . 9$$

and as mentioned above Ln (dS/dt) against t gives

$$Slope = - ( {k}_{endo} [ P ] + {k}_{exo} ) , \\ Intercept = Ln ( {k}_{endo} [ P ] {S}_{max )} ) at Ln \frac{dS}{dt} = 0. \qquad Eq . 10$$

Assumption: This analysis does not take account of particle re-uptake after exocytosis.

Flow cytometry and ELISA.

CD44+CD8+ cells and

CD4+CD25+Foxp3+ Tregs were acquired at 3- and 5-day post CY treatments for CY-induced mice. For spontaneous T1D animal model, T cells were collected at day 1 after the last NP dose. In both cases, pancreatic lymph nodes were harvested and processed using a 40 μm cell strainer. Cell surface markers were ascertained with fluorescent antibodies for CD8 (APC), CD44 (PE), CD4 (APC), and CD25 (Alexa Fluor-700) by incubating for 30 min at 4° C. Cells were then fixed, permeabilized, and stained for Foxp3 (PE) using the Foxp3 staining kit (eBiosciences). After the final wash, samples were immediately run on an LSR-II multicolor flow cytometer (BD Biosciences) and analyzed using FlowJo software (Tree Star). To study antigen-specific T cell responses, OVA-specific CD4+ cells were used in OTII co-culture assays. BMDCs (2.5×10⁴ cells/well, 96 well plate) were pretreated with pUDCA NPs for 24 h, washed, and then stimulated with LPS (10 ng/mL) and OVA (20 μg/mL) for 24 h, followed by co-culture with DTII CD4+ T cells (5×10⁴ cells/well, 96 well plate) for 3 days. Cell proliferation and cytokine production were then quantified.

BMMs (10⁵ cells/well, 96 well plate) were incubated with pUDCA NPs (50 μg/mL), UDCA monomer (50 μg/mL), PLGA (50 μg/mL), or PBS for 4 h and added with CpG (100 ng/mL). After 20 h, media supernatant was collected for IL-6, IL-10, and CCL1 readouts. Cells were stained for F4/80 (Alexa Fluor-647), CD86 (PE), and CD206 (FITC). After 3 washes with buffer (2% FBS in PBS), samples were fixed in 2% paraformaldehyde and run on an Attune N×T multicolor flow cytometer (Life Technologies). CD11c-F4/80+NP+ was used for NP tracking of macrophages with dye-loaded particles. The NP+ designation refers readout of the particle via fluorescence of loaded dye. Mice were fasted for 4 h and treated with labeled pUDCA NPs by oral gavage (500 mg/kg, 250 μL). After one day, pancreas, liver, lungs, and spleen were harvested and gently processed with a homogenizer and using a 40 μm cell strainer and plunger for separations of cells from debris. Cell surface markers were stained with fluorescent antibodies for F4/80 (Alexa Fluor-700) and CD11c (PE-Cy7) and measured by Attune N×T multicolor flow cytometer. All antibodies were diluted 1:500 for flow cytometry and cytokines (IL-6, IL-10, and IFNγ) were measured by ELISA (BD Biosciences).

Biodistribution and Histology.

B6, NOD, or Nude mice were fasted for 4 h and treated with DIR- or coumarin 6-encapsulating NPs by oral gavage (50, 100, or 500 mg/kg, 250 μL). Free DIR or coumarin 6 (solubilized with 1% Tween 20) served as controls. Mice were sacrificed at time points of 4, 8, 12, or 24 h post gavage, and a Bruker molecular imaging instrument (Carestream Health, Inc.) was used to scan organs ex vivo to measure fluorescence intensity. Fluorescence data was fit to the one component exponential decay model: Y=Y_{f}+(Yₒ−e^{-kt}). DIR-loaded NPs formulated by pUDCA, PLGA or the mixture (50:50, w/w) were also intravenously (IV) administered (100 mg/kg, 50 μL) to mice via tail vein injection to compare their biodistribution with free dye. Macrophage Depletion Kit (Clodrosome®, Encapsula NanoSciences, 100 mg/kg, intraperitoneal (IP) injection) was used to deplete macrophages in B6 mice. For histology, pancreata from the mice orally received iron oxide-loaded pUDCA NPs were fixed in 10% neutral buffered formalin for histological analysis by hematoxylin and eosin (H&E) and Prussian blue stains. The stained sections were prepared by the Yale University Pathology Histology Service. Tissues were imaged on a Nikon TE-2000U microscope with a Nikon DS Fi1 color camera and NIS Elements AR software (version 2.30).

Experiments with Diabetic Animal Models.

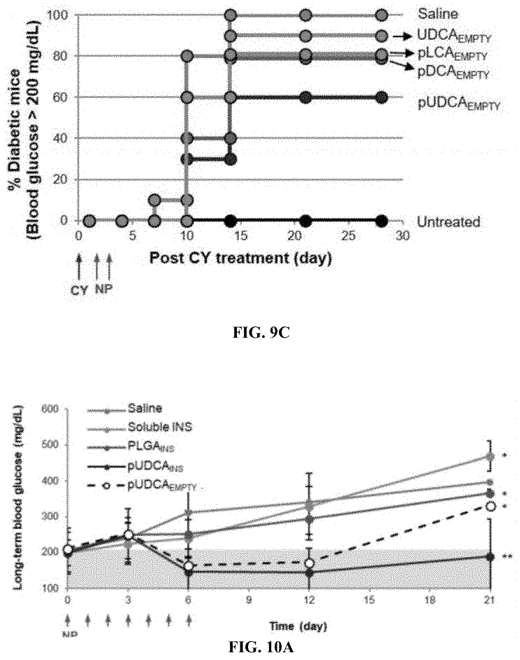

NOD mice were intraperitoneally injected with CY (200 mg/kg) to induce acute type I diabetes (T1D) (FIG. 9A). After 24 h, the mice were orally gavaged with empty NPs, RAPA-loaded NPs (50, 100, and 500 mg NP/kg=40 mg RAPA/kg), soluble RAPA (40 mg/kg solubilized with 1% Tween 20), and saline. pUDCARAPA was orally administered at day 1 (Dose I) or twice on day 1 and 2 (Dose II). Blood glucose level was monitored using a blood glucose monitor (TRUERESULT® meter, Home Diagnostics, Inc.). Two readings (1 day apart) higher than 200 mg/dL were taken as an indication onset of T1D. pUDCA_{RAPA} was also compared to “Gold Standard” insulin administration, insulin administered subcutaneously (SQ) or intraperitoneally (IP) using this model. After 7 consecutive injections of insulin or oral gavage of pUDCA_{INS} with an equivalent insulin dose, blood glucose was measured over the course of 25 days. Data was fitted using an operational receptor depletion model, Y=Basal+(Effectₘₐₓ/Basal)/(1+operate), where operate=(((10^{logkA})+(10^{X}))/((10^{(logtau+X)}))ⁿ, Effectₘₐₓ is the maximum possible system response, Basal is the response in absence of agonist, kA is the agonist-receptor dissociation constant, and tau is the kinetics of lowering to half-maximal response.

For spontaneous T1D model, NOD mice were housed for approximately 2 months to allow them to naturally develop T1D. For spontaneous T1D model, NOD mice the same age (8 week old) were chosen for the study only when they became diabetic in the same week (16 week old, after 2 random tail vein blood glucose measurements of 200±15 mg/dL). The mice were orally treated with empty NPs, INS-loaded NPs (100 or 500 mg NP/kg and 285 mIU INS/kg) or free INS (285 mIU/kg) 7 times for 1 week to monitor glycosuria and body weight. Concentration of GLP-1 was measured using an ELISA kit (Invitrogen) from plasma. Swine were fasted for >12 hours with access to water and alloxan was administrated IV (250 mL, 150 mg/kg). At 10 day post alloxan treatment, the animals (n=3) were orally gavaged with pUDCA_{INS} (6.4 mg/kg equivalent to 100 mg/kg in mice) 7 times daily and monitored glucose readings using a continuous glucose monitoring system, DexCom 6 (Dexcom. Inc.). Calibration was done by ear stick with Relion Prime BG Meter (Relion) and further venous calibration was performed from whole blood samples by Antech Diagnostics.

Calculations for INS Dose Chosen in the Spontaneous TID Study.

It is known that 1 international unit (IU) of INS is needed to drop blood glucose level of 50 mg/dL for human. To lower high blood glucose level (>400 mg/dL) below hyperglycemia threshold (200 mg/dL), at least 4 IUs of INS is required. That is 0.02 IUs of INS for mice according to the following equation (9).

$$HED = Animal NOAEL \times {( \frac{Weight of animal ( kg )}{Weight of human ( kg )} )}^{( 1 - 0.67 )} \qquad Eq . 11$$

where HED=human equivalent dose (mg/kg), Animal NOAEL=No Observed Adverse Effect Levels for animal (mg/kg), 1 IU of INS=0.036 mg based on the World Health Organization conversion factor.

The loading of INS in pUDCA NPs was approximately 20 ng/mg of NP and it is 5.6×10⁴ IU/mg of NP. In order to feed 0.02 IUs of INS, 35 mg of pUDCA_{INS} NPs should be treated to single mouse. Therefore, diabetic mice were treated with 3 doses of 10 mg of NPs and continued to treat the mice with additional 4 doses to keep the glucose level low (total 0.04 IUs of INS and 70 mg of NPs).

Toxicology of pUDCA and UDCA.

Acute toxicity studies were performed with 10-weekold, female B6 mice. Mice were orally dosed with pUDCA (100 and 500 mg/kg) and UDCA (100 mg/kg) at day 0. Kits from Teco diagnostic were used at day 3, 5, and 7 for analysis of serum concentrations of alkaline phosphatase, alanine transferase, total bilirubin, and blood urea nitrogen concentrations. EDTA anticoagulated blood was analyzed by a Hemavet blood counter (Drew Scientific).

Statistics.

All statistical analyses were performed using GraphPad Prism software (version 7.01). Experimental comparisons with multiple groups used ANOVA analysis with Bonferroni's post test or Two-tailed Student's t tests were performed. Log-rank test and χ² statistical analysis was performed for survival data. A P value of 0.05 or less was considered statistically significant.

Results

Fabrication of Bile Polymer Solid Biodegradable NPs.

To create BA carriers, biodegradable pBAs were first synthesized then the polymers were formulated into NPs. This methodology, as opposed to the use of individual BA monomers, was for four reasons: 1) To increase stability of the BA drug combination (if present) during digestive transit in comparison to micellar vesicles that form above the BA micelle concentration. 2) To formulate carriers that can deliver an encapsulated drug (if present) in a sustained fashion. 3) To ensure a constant ratio of BA to encapsulated drug (if present) during the delivery and release process. 4) To increase the valency of the BA for potential increased avidity binding to BA receptors.

Polymerization of BAs and Formulation into NPs.

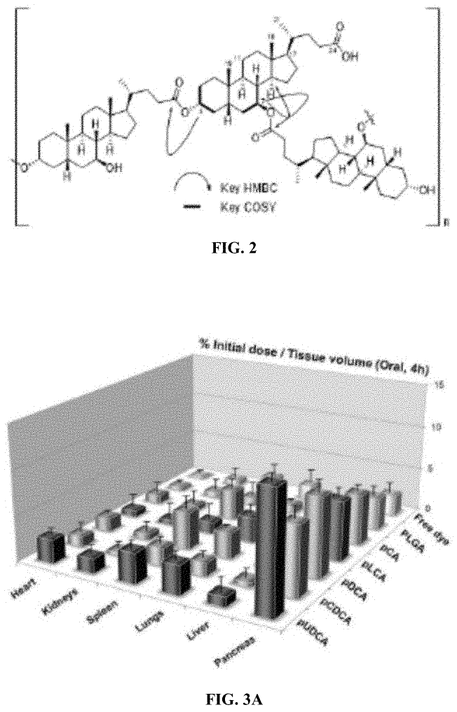

For screening purposes, a panel of BAs which included cholic acid (CA), chenodeoxycholic acid (CDCA), deoxycholic acid (DCA), lithocholic acid (LCA), and ursodeoxycholic acid (UDCA) was tested (FIGS. 1A-1E). BAs were polymerized under mild conditions, 40° C. and atmospheric pressure for 2 h (see METHODS). Use of diisopropyl carbodiimide (DIC) and a 1:1 salt of dimethylaminopyridine and p-toluenesulfonic acid (DMAP/PTSA) led to selective activation and esterification at the carbon-24 position of the BAs yielding BA polymers (FIGS. 1F-1J). Polymerization proceeded from the negatively charged monomer termini. The extent of polymerization and molecular weight were ascertained by nuclear magnetic resonance (NMR) and gel permeation chromatography (GPC), respectively. Polymerization and crosslinking were validated by two-dimensional (2D) heteronuclear single-quantum coherence (HSQC) spectroscopy and 2D double-quantum-filtered correlation spectroscopy (DQF-COSY) analyses (FIG. 2). In general, the number average molecular weight (Mn) range was 1360-2230 g/mol, and the weight average molecular weight (Mw) was in the range 1600-3210 g/mol. Polymer dispersity was assessed with a polydispersity index (PDI) and did not exceed 1.5 (Table 5). Linearity of the polymer was assessed by 2D NMR where it was shown that two hydroxyl substituents at C-3 and C-7 were esterified with 2.5:1 molar ratio during the polymerization process.

A water-in-oil-in-water (W/O/W) double-emulsion methodology was used for nanoparticle formulations (FIGS. 1F-1J). The polyester, PLGA, was used here as a comparator for drug delivery. Blends of pBA and PLGA were fabricated to demonstrate the relative impact of pBA on PLGA function. Encapsulation of agents was achieved by first initial dissolution of the polymer in chloroform then during emulsion formulation agents were added. The agents utilized in this study include: infrared fluorescent dye (1,1′-dioctadecyl-3,3,3′,3′-tetramethylindotricarbocyanine iodide (DIR), green fluorescent dye (coumarin 6), the drug rapamycin (RAPA), 10-20 nm iron oxide particles used for histological staining with the iron sensitive Prussian blue, and saline buffered mouse insulin (see METHODS). Loading efficiencies of DIR, coumarin 6, RAPA, iron oxide, and insulin were 6.7, 84.3, 80.1, 9.0, and 0.02 μg per mg of particles, respectively. Spherical morphology of the NP was validated by scanning electron microscopy (SEM) which showed uniformly spherical particles with an average diameter of 344.3±4.7 nm quantitated using dynamic light scattering (DLS, Zetasizer, Malvern Instruments) (Table 1). The pBA NPs electrostatic charge was negative with a zeta-potential of −24.9±4.4 mV. All preparations were engineered to have the similar size, charge, morphology and encapsulation efficiency to ensure that any observed biodistribution or functional differences were strictly a function of intrinsic material properties and not biophysical variations (Table 1).

TABLE 1
Particle properties and loading efficiency.

| | | | | Mean diameter | | Zeta-pot-al | Dye loading | Loading efff-y |
|---|---|---|---|---|---|---|---|---|
| | Mn^{a} | Mw^{b} | PDI^{c} | (nm) | PDI^{d} | (mV) | (%)^{e} | (%)^{f} |
| PLGA | 2451 | 4184 | 1.707 | 328.8 ± 3.4 | 0.304 | −27.5 ± 2.8 | 0.699 ± 0.045 | 69.9 ± 4.5 |
| pCA | 1972 | 2962 | 1.502 | 360.3 ± 11.2 | 0.296 | −24.6 ± 3.1 | 0.702 ± 0.012 | 70.2 ± 1.2 |
| pLCA | 1357 | 1598 | 1.177 | 337.9 ± 21.0 | 0.276 | −27.1 ± 10.4 | 0.730 ± 0.020 | 73.0 ± 2.0 |
| pDCA | 1842 | 2523 | 1.370 | 311.9 ± 24.1 | 0.213 | −22.7 ± 1.5 | 0.687 ± 0.064 | 68.7 ± 6.4 |
| pCDCA | 1741 | 2284 | 1.312 | 335.1 ± 9.8 | 0.011 | −27.8 ± 10.1 | 0.687 ± 0.014 | 68.7 ± 1.4 |
| pUDCA | 2225 | 3210 | 1.443 | 344.3 ± 4.7 | 0.164 | −24.9 ± 4.4 | 0.674 ± 0.005 | 67.4 ± 0.5 |
| Blend | — | — | — | 299.5 ± 14.3 | 0.131 | −22.2 ± 5.6 | 0.726 ± 0.019 | 72.6 ± 1.9 |
| (pUDCA/ | | | | | | | | |
| PLGA)^{g} | | | | | | | | |

^{a}The number average molar mass (GPC)
^{b}The weight average molar mass (GPC)
^{c}Polymer polydispersity index (GPC)
^{d}Particle polydispersity index (DLS)
^{e}(weight of encapsulated dye/weight of NPs) × 100
^{f}(weight of encapsulated dye/weight of dye used for encapsulation) × 100
^{g}Polymer blend nanoparticles (pUDCA:PLGA = 50:50, w/w).

Improved GI Transport: Stomach Protection and Enhanced Intestinal Permeation.

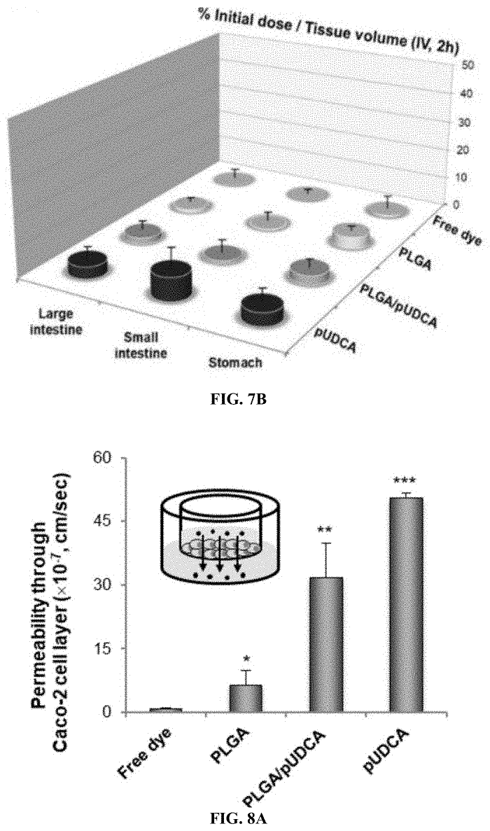

BA ionization under acidic conditions impact its water solubility, limiting water penetration into individual particles while promoting hydrophobic interactions between particles in acidic conditions. This protective mechanism, which limits exposure of the majority of particles to the stomach milieu, reverses with increased pH conditions in the intestinal lumen. Under simulated stomach conditions, the release of dye from pUDCA was compared to the release from a well-established delivery vehicle, PLGA alone, PLGA modified with an anionic protective copolymer of methacrylic acid and acrylic acid, (EUDRAGIT®®) or PLGA blended with pUDCA. PLGA particles began to degrade at 2 and 4 h post incubation, releasing up to 8% of the encapsulated dye. In contrast, pUDCA retained dye over 4 h in a manner similar to EUDRAGIT® modified PLGA (FIG. 7A). PLGA blended with pUDCA imparted a EUDRAGIT® comparable stability which further validated the pH responsive properties of pUDCA. The spherical particulate morphology of pUDCA was also retained while that of PLGA showed significant swelling over time in acidic conditions (FIG. 4F). Blending PLGA with pUDCA had a transient stabilizing effect. These observations speak to the protective nature of pBAs, which impart stability on encapsulated insulin during GI trafficking (FIG. 7B).

Following stomach digestion, NP encounter a higher pH microenvironment and become more water permeable. BA particle permeation through the intestinal lumen can take place via passive transcytosis through intestinal epithelial cells or active transport via engagement of colonic receptors. pUDCA appears to transport passively through the intestinal lumen since the permeability of dye-loaded pUDCA NPs through a model epithelial cell line (Caco-2 human cell) was significantly more than PLGA, PLGA blended with pUDCA, or other pBA NPs (FIG. 4H). Indeed, the permeability coefficient of pUDCA is approximately 50×10−6 cm/sec which means that it will be fully absorbed in the intestinal lumen in humans.

Blood pool entry of BAs can be mediated by either enterohepatic circulation or blood cells such as monocytes or macrophages in the intestinal lumen. To elucidate the mechanism to pUDCA NP circulation, the NP were intravenously (IV) injected and found that pUDCA NPs showed the high pancreatic uptake profile at 2 h post IV administration. Pancreatic accumulation thus also involved cell transport in the blood and was not entirely driven by enterohepatic circulation. Given that intestinal macrophages are one of the largest pools of cells in the body and are in immediate proximity to the lamina propria in the healthy colon, a reduction in the number of cells in this pool with depleting agents such as clodronate liposomes should affect pancreatic biodistribution. The effect on biodistribution is shown in FIG. 4I, supporting the partial role of macrophages in particle biotransport to the pancreas. Complete elimination of pancreatic distribution via this mechanism would negate the role of enterohepatic circulation, but FIG. 4J shows that only 16% of macrophages were associated with pUDCA NPs. The overlapping mechanisms contributing to NP circulation are summarized as follows. Here, NP permeating the intestinal epithelium enters the duodenum and can access the pancreatic duct via the common bile duct. Particles may also be uptaken by resident and circulating monocytes and macrophages to traffic to pancreas, or on their own percolate through capillaries and lymphatic vessels with and without a cellular-host. Another potential mechanism is binding to serum albumins which have affinity to different bile acids.

pUDCA NPs Bind the Extracellular Bile Acid Receptor (TGR5) with High Avidity and Facilitate Glucagon-Like Peptide and Endogenous Insulin Secretion.

BAs engage apical membrane transporters such as CD36, caveolin, and fatty-acid transporter (FAT) to actively transport fatty foods, thus high avidity binding can enhance endocytosis and BA-mediated signal transduction cascades. Furthermore, monomeric UDCA is a weak TGR5 receptor agonist, thus understanding the changes in binding affinity would help elucidate the amplified response of polymeric BA compared to monomer. The competitive binding of pUDCA to macrophages saturated with an anti-TGR5 antibody is shown in FIG. 4K and dissociation constants (k_{d}) values shown in Table 2.

TABLE 2
Dissociation constants for pUDCA, UDCA, and PLGA.

| | Specific kd | Non-Specific kd |
|---|---|---|
| pUDCA | 1.25 | −0.0061 |
| UDCA | 29.97 | −0.0055 |
| PLGA | N/A | ~0 |

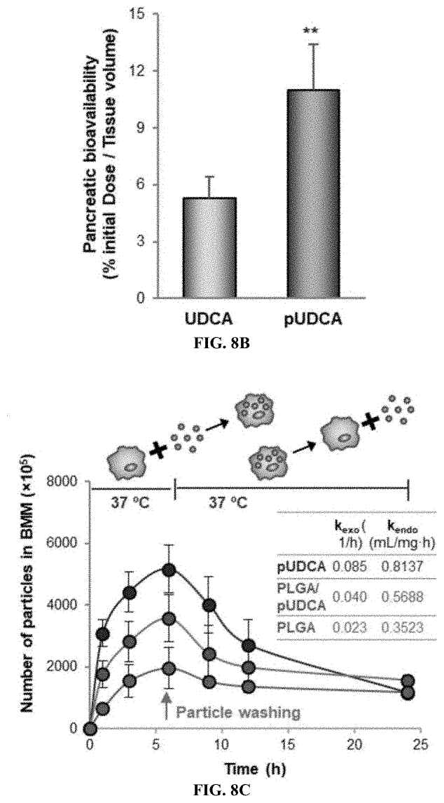

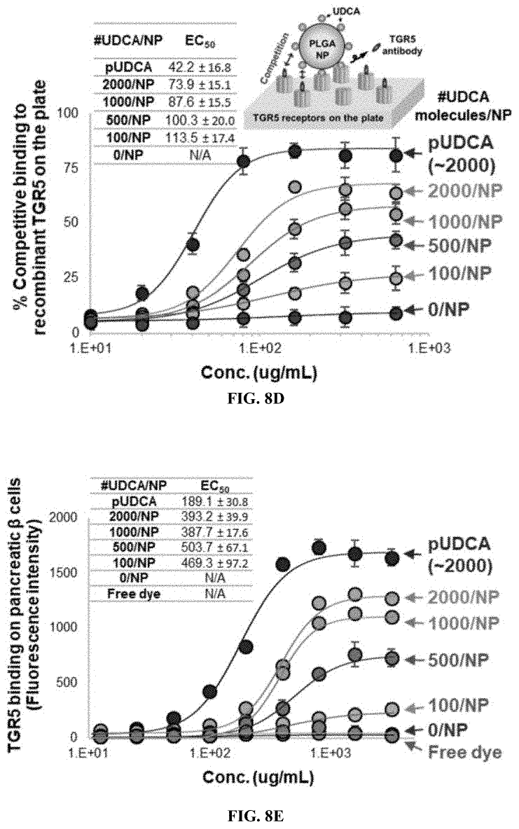

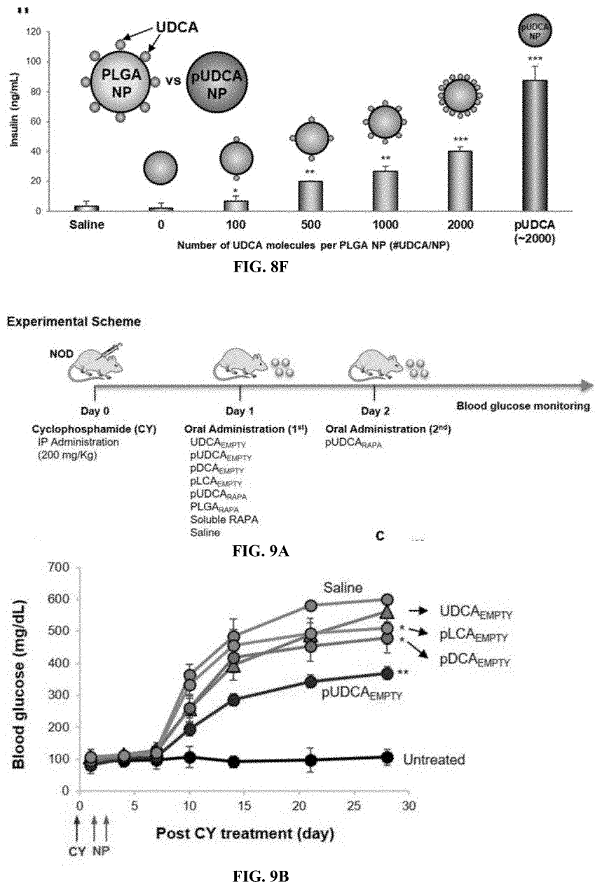

Compared to UDCA, the affinity of pUDCA was about 30 fold greater with minimal non-specific binding (Table 1). PLGA NP used as a negative control showed no affinity. Rates of intracellular NP trafficking are also impacted by higher binding avidity. A quantitative assessment of pUDCA NP rate of endocytosis and exocytosis in macrophages at 37° C. is shown in FIG. 8E and Table 3.

TABLE 3
Rate of endocytosis and exocytosis in macrophages at 37° C.

| | Kₑₓₒ | K_{endo} |
|---|---|---|
| | (1/h) | (ml/mg · h) |
| pUDCA | 0.085 | 0.8137 |
| PLGA/pUDCA | 0.040 | 0.5688 |
| PLGA | 0.023 | 0.3523 |

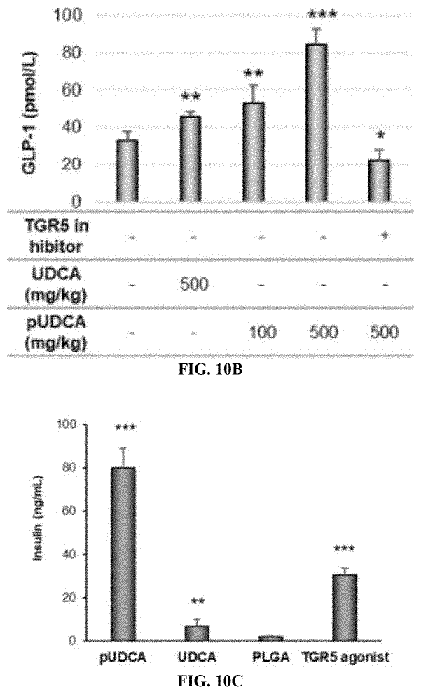

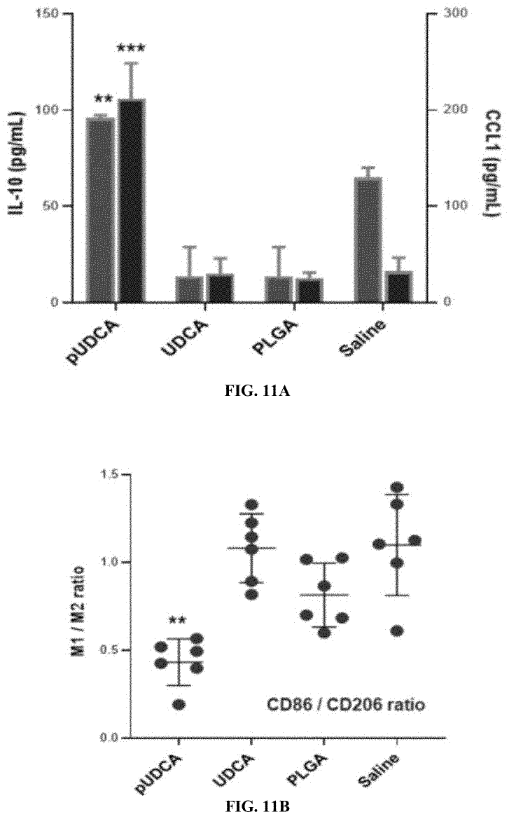

The rate of internalization, (k_{endo}), was 3.6 fold higher compared to non-binding control (PLGA) and the rate of exocytosis, (kₑₓₒ), was 2.3 fold faster. The assay validity in this quantitative determination of rates was performed at 4° C. where minimal or no active internalization or exocytosis is shown in FIG. 4L. Elevation in the magnitude of internalization or faster exocytosis depended on the amount of pUDCA in the formulation. A 50% reduction in the pUDCA concentration, by blending with PLGA, led to an internalization rate or exocytosis rate that was greater or faster respectively compared to PLGA itself. Thus, increased binding avidity of pUDCA led to increased rates of receptor-mediated intracellular transport (endocytosis and exocytosis). Indeed, high avidity binding was not exclusive to only macrophages but was also observed in pancreatic β cells where TGR5 binding results into enhancements in endogenous GLP-1 secretion (FIG. 10B) and insulin production (FIG. 10C) facilitating effective control over blood glucose levels. In addition, to metabolic control, increased binding avidity also makes possible an intrinsically potent anti-inflammatory immune response control by engagement of multiple immunomodulatory machineries, for example, through macrophage phenotypic skewing from M1 to M2 (FIG. 11A), reduction of IFNγ secretion (FIG. 4D), reduction of pro-inflammatory IL-6 (FIG. 4E), augmentation of anti-inflammatory activity with production of IL-10 and CCL1, and induction of tolerance via regulatory T cell response (CD4+Foxp3+CD25) proliferation in parallel with suppression of the activated CD8+(CD44+ CD8+) T cell response. This multivariate innate and adaptive immune modulation by the carrier material itself offers significant therapeutic control over pancreatic inflammation in conjunction with provisioning more than additive drugs towards neutralization of the pro-inflammatory response associated with hyperglycemia.

pUDCA Effects are Mediated Through Enhancement in Valency and Proximity of Multivalent Display

The anti-inflammatory effects of UDCA have been widely studied and exploited medicinally for dissolution of gallstones and prevention of chronic graft versus host disease in the liver. In Chinese, the word “Urso” means “bear” and ursodeoxycholic acid is a multibillion-dollar industry in Chinese traditional medicine, owing to its anti-inflammatory properties and general benefits in healthy and sick populations. Additionally, clinical trials are ongoing focusing on UDCA's role in treatment of diabetes. pUDCA is a multivalent form of UDCA that owes its enhanced effects to high avidity binding due to spatial density of the UDCA on the surface of the particles to the TGR5 receptor in addition to functioning as a carrier.

To demonstrate that increased multivalency is the mechanism behind the observed effects, the pUDCA effect on insulin secretion in vitro from pancreatic β cells was tested. PLGA, a carrier with no intrinsic effect on insulin secretion from pancreatic cells, was modified with different concentrations of monomeric UDCA up to full saturation of the surface which was approximately 2000 molecules of UDCA per PLGA particle. Tables 3 and 4 show the dose at which 50% lowering of TGR5 antibody fluorescence is achieved as a function of different UDCA densities on the surface of PLGA in comparison to pUDCA. The EC50 was calculated based on curve fitting the binding data to a competitive binding isotherm as discussed in the methods). See Tables 4 and 5.

TABLE 4
EC₅₀ values for binding of pUDCA and PLGA nanoparticles
with different concentrations of monomeric
UDCA on plate-immobilized TGR5.

| # UDCA/NP | EC₅₀ |
|---|---|
| pUDCA | 42.2 ± 16.8 |
| 2000/NP | 73.9 ± 15.1 |
| 1000/NP | 87.6 ± 15.5 |
| 500/NP | 100.3 ± 20.0 |
| 100/NP | 113.5 ± 17.4 |
| 0/NP | N/A |

TABLE 5
EC₅₀ values for binding of pUDCA and PLGA nanoparticles
with different concentrations of monomeric UDCA
on TGR5 on pancreatic β cells.

| # UDCA/NP | EC₅₀ |
|---|---|
| pUDCA | 189.1 ± 30.8 |
| 2000/NP | 393.2 ± 39.9 |
| 1000/NP | 387.76 ± 17.6 |
| 500/NP | 503.7 ± 267.1 |
| 100/NP | 469.3 ± 97.2 |
| 0/NP | N/A |
| Free dye | N/A |

While an increase in UDCA concentration on the surface potentiated the binding of a non-pUDCA carrier (i.e. PLGA) to target pancreatic cells, the effect of pUDCA was still superior to PLGA particles saturated with monomeric UDCA (up to 2000 UDCA molecules per PLGA particle). Thus, the enhancement effect cannot be fully recapitulated by increasing only the monomeric valency on a nanoparticle. Additional geometrical or biophysical factors are required, and these can range from the proximity of the molecules on the multivalent platform, to mobility or relative persistence of ligands on the activating surface. Consistent with the notion that increased affinity alone is often not a sufficient parameter to achieve optimal responses, the spacing between proteins needs to be accounted for the design of multivalent targeting systems. Polymerized ligands can inherently span the lengths for targeting BA receptors because the composition is a distribution of randomly spaced ligands that span a wider set of length scales in comparison with monomers conjugated or adsorbed to the surface of a solid support or nanoparticle. In addition, pUDCA encapsulating agent allows for the provision of a fixed ratio between bile acid and released drug over a time span needed to synergize both moieties for improved response. The surface of a particle decorated with monomer will be degraded with kinetics that are different from the release kinetics of the encapsulated agent. If combination delivery of a pair of pluripotent drugs is required for therapy, then the temporal stability of the combination plays a critical role. Temporal stability is a pre-requisite to potential more than additive activity and that would be best achieved via a system that provisions the active drugs at a fixed ratio over time. This can be accomplished if the carrier is one of the two effector drugs (the bile acid and encapsulated agent).

The multivariate properties of this carrier are summarized as follows. After oral ingestion (pH-7-7.5), particles are anionic and dispersible. Protonation in the stomach (pH 2-3) limits water penetration. The small intestine pH (6-6.5) reduces hydrophobic interactions and enable water penetration in particles, and transport through intestinal lumen, followed by binding and internalization by intestinal macrophages.

In summary, particles circulate systemically by a number of mechanisms, involving either cell-mediated transport or particle trafficking alone from the duodenum to the common bile duct facilitated entry into the exocrine pancreas. The particles show protective and localized transport through the GI tract, metabolic and immunological control over the response. Together these properties are a means to potentiate the effects of an encapsulated anti-inflammatory or metabolic therapeutic.

### Example 2. Post-Oral Ingestion pBA NPs Preferentially Accumulate in Pancreatic Tissue

Materials and methods are described above.

Results

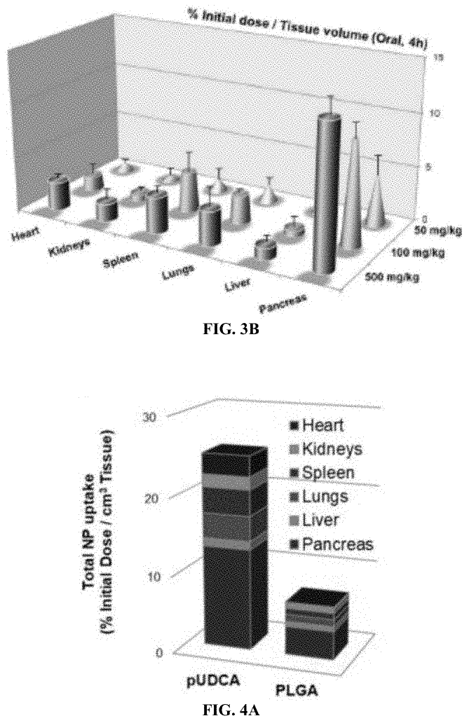

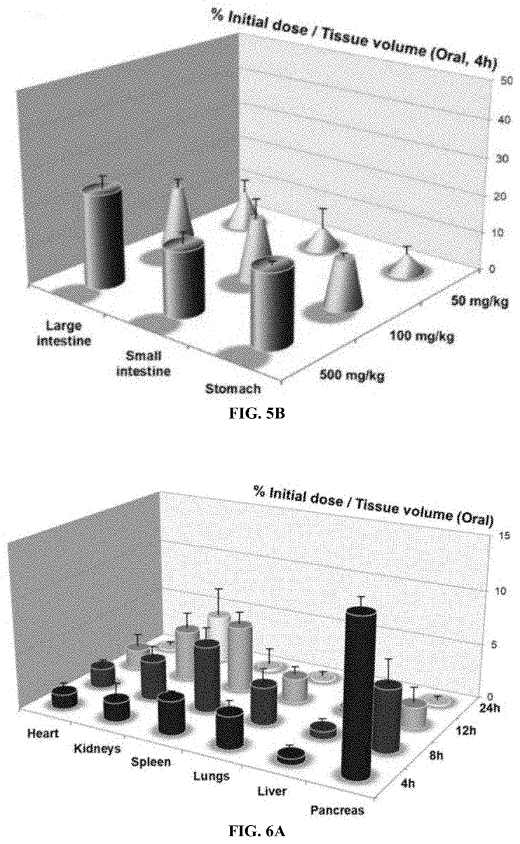

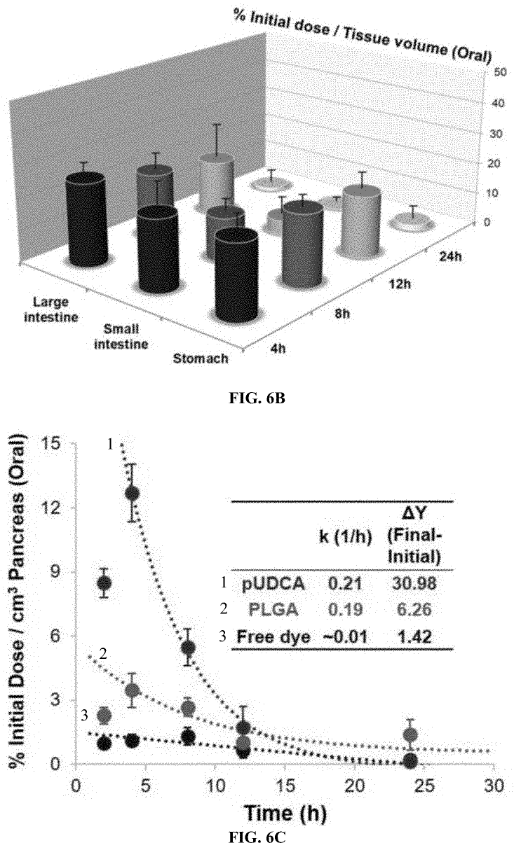

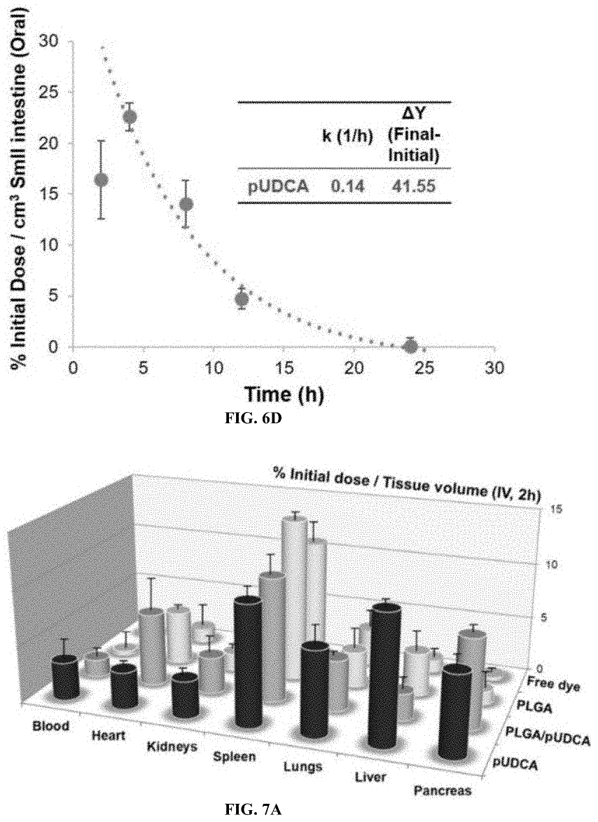

Initial studies examined if pBA NPs intrinsically distributed differently to organs after oral gavage. Biodistribution in animals was conducted using fluorescent dye loaded pBA NPs. Animals were first fasted for 4 h prior to the experiment then orally gavaged with the NP formulations in saline followed by resumption of normal food and water. At 4 h post gavage, animals were euthanized, and organs were harvested to be fluorescently imaged. Quantitative fluorescence was conducted with a multispectral imaging platform (Bruker multispectral MS FX PRO imaging system). It was observed that, in contrast to control PLGA NP, pBA NPs were retained to a higher magnitude in organs and especially pancreatic tissue (FIGS. 3A and 4A). In the GI, the increased fluorescence levels at 4 h post NP ingestion (FIG. 5A) was due to digestive kinetics in the stomach and intestines, and not steady state biodistribution accumulation, which is what occurred with pancreas. While pUDCA showed at least five fold greater pancreatic accumulation compared to controls and its biodistribution was dose-dependent (FIGS. 3B and 5B), clearance rate of pUDCA was similar to PLGA (FIGS. 6A-6D and Tables 6 and 7).

TABLE 6
Clearance rate of particles in the pancreas after oral administration.
% Initial Dose/cm³ Pancreas (Oral)

| | k (1/h) | ΔY (Final-Initial) |
|---|---|---|
| pUDCA | 0.21 | 30.98 |
| PLGA | 0.19 | 6.29 |
| Free dye | ~0.01 | 1.42 |

TABLE 7
Clearance rate of pUDCA in the small
intestine after oral administration.

| | k (1/h) | ΔY (Final-Initial) |
|---|---|---|
| pUDCA | 0.14 | 41.55 |

This indicated that a higher pUDCA flux to pancreas and subsequent slower accumulation was the rationale for accumulation and not slower clearance rate; a fact qualitatively supported by strong fluorescence intensities in excised pancreata at 4 h post oral gavage.

The preferential accumulation in pancreata may be a function of the encapsulated agent, which may impact particle biodistribution and pharmacokinetics. To understand the role of loaded agent on particle flux to organs, identical biodistribution studies were performed, but this time with a different dye (coumarin 6, Log P=1.8), which is more hydrophilic than DIR (Log P=17.4). If particle flux to the pancreas changes as a function of the hydrophobicity of the encapsulated dye, then the observations reflect on the nature of the drug physicochemical properties and its biodistribution in the body. After repeating similar biodistribution experiments with coumarin 6, fold increase in the pancreatic accumulation compared to PLGA was similar to that of DIR, conclusively proving that level of pancreatic accumulation of NP was independent of the physiochemical properties of the loaded agent (FIG. 4B), but dependent on the particle composition.

To examine the microlocalization of the pUDCA NPs in pancreatic tissue, stained excised pancreata were histologically with Prussian blue after oral administration of iron oxide loaded pUDCA NPs. Prussian blue is sensitive to the iron content in histological tissue sections. Particles were found in and around exocrine pancreatic acinar cells and less with endocrine cells or capillaries. There were no observable toxic side effects due to this accumulation since H&E staining showed normal cell morphologies with no infiltration of leukocytes.

Since pancreatic exocrine cells are proximal to the duodenum with the common bile duct emptying there, but also passing through the pancreatic head, one likely mechanism for pUDCA trafficking to the pancreas after oral ingestion could be enterohepatic cycling from the duodenum. Supporting the enterohepatic trafficking mechanism, fluorescence images of DIR loaded pBAs including pUDCA, in most cases, showed greater fluorescence intensity in the head region of the pancreas compared to the tail. Furthermore, dye loaded in pUDCA NP also accumulated in the gall bladder showing that the transport mechanism may have been partially mediated by enterohepatic circulation. Finally, pancreatic accumulation was not a strict function of mouse genetics as shown three different mouse clones showed the similar pattern of biodistribution profiles.

Comparative toxicology was performed between the monomer UDCA and polymer pUDCA (FIG. 4C). Macrophages and Caco-2 cells were tested with pUDCA NPs (1 mg/ml equivalent to 1000 mg/kg in mice). There was no obvious effect on the cell viability. Importantly, when organ level clinical toxicology was performed with oral doses of UDCA (500 mg/kg) and pUDCA (100 and 500 mg/kg) there was similarly no observable effects on organ specific function both for monomer and polymer. Hepatotoxicity assessed by serum levels of alkaline phosphatase and alanine aminotransferase, renal toxicity monitored by blood urea nitrogen levels, hematological toxicity monitored by the hematocrit, leukocyte counts, platelet counts, and hemoglobin content were all within normal physiological ranges.

FIGS. 7A and 7B show high pancreatic accumulation of IV injected pUDCA NP, which was similar to the observations with orally gavaged pUDCA particles, indicating that pancreatic accumulation involved transport by macrophages cells and/or serum albumins, which are critical components governing biodistribution and clearance of foreign particles in the blood. Pancreatic accumulation thus was not entirely driven by enterohepatic circulation.

The mechanisms of pUDCA biodistribution, pancreatic accumulation, and immune modulation after oral ingestion show that after oral ingestion (pH-7-7.5), particles are anionic and dispersible. Protonation in the stomach (pH 2-3) limits water penetration. The small intestine pH (6-6.5) reduces hydrophobic interactions and enable water penetration in particles, and transport through intestinal lumen, followed by binding and internalization by intestinal macrophages. In summary, particles circulate systemically by a number of mechanisms, involving either cell-mediated transport or particle trafficking alone from the duodenum to the common bile duct facilitated entry into the exocrine pancreas.

### Example 3. pUDCA is a Strong Agonist of the Extracellular Bile Acid Receptor TGR5

Given that UDCA has established metabolic and immunologic regulation functions despite being a weak agonist of extracellular BA receptors; specifically, the G-protein coupled receptor (TGR5), it was tested whether polymerization of UDCA would greatly amplify its function but in addition allow for parallel function of the system as an insulin carrier and the potential for signal amplification to function in more than additive effect with insulin delivery and ultimately, its effector response.

Materials and methods are as described above.

Results

The observed potency with pUDCA may be due to the multivalent nature of the pBA platform facilitating greater BA receptor agonism compared to monomer and hence a greater effector response. Previous reports have shown that UDCA is a weak agonist of the extracellular TGR5 BA receptor. It was tested whether the increased valency introduced via polymerization directly impacts TGR5 activation. To examine this effect and its impact on more than additive effect with insulin in reduction of blood glucose levels, empty pUDCA and UDCA were tested. UDCA was administered at the highest dose (500 mg/kg) and compared to pUDCA at the same dose in blood glucose level reduction. TGR5 activation by pUDCA was validated by pre-treatment of a cohort of animals with a TGR5 inhibitor (Triamterene, 50 mg/kg, IP), 1 h before oral gavage of pUDCA. FIG. 10B compares the effect of material alone (i.e., empty UDCA and pUDCA) in reduction of glucose levels in diabetic mice over the seven day period. Not only was pUDCA exceptionally potent, exhibiting greater reduction in contrast to UDCA, but this affect appeared to be TGR5 mediated since inhibition of TGR5 in vivo abrogated the metabolic response. The binding and activation of pUDCA compared to UDCA was investigated (FIGS. 8A-8F). Here, these results show the amplified effector function of the material on its own, at least in the short-term (7 days), and potential more than additive effect with loaded insulin in blood sugar level control (FIG. 10A).

### Example 4. pUDCA is a Carrier and a More than Additive Metabolic/Immunomodulatory Drug

Materials and Methods

Materials and Methods are as described above.

For testing insulin production from pancreatic (3 cells promoted by activation of TGR5 receptor, the mouse pancreatic (3 cell line (MIN6, ATCC) cells were incubated in Hank's balanced salt solution (HBSS, Life Technologies) containing 3 mM glucose for 2 h and then for 30 min in HBSS with 25 mM glucose and UDCA, PLGA or pUDCA NPs (40 μg/mL). Concentration of insulin was measured using an Ultrasensitive Insulin ELISA kit (ALPCO).

The same experiment was performed in the presence of TGR5 antagonist, triamterene (50 μg/mL) as a control to differentiate inherent insulin production from the cells without TGR5 activation and used to normalize the results.

For testing insulin production from pancreatic β cells promoted by activation of TGR5 receptor, the mouse pancreatic β cell line (MIN6, ATCC) cells were incubated in Hank's balanced salt solution (HBSS, Life Technologies) containing 3 mM glucose for 2 h and then for 30 min in HBSS with 25 mM glucose and UDCA, PLGA or pUDCA NPs (40 μg/mL). Concentration of insulin was measured using an Ultrasensitive Insulin ELISA kit (ALPCO). The same experiment was performed in the presence of TGR5 antagonist, triamterene (50 μg/mL) as a control to differentiate inherent insulin production from the cells without TGR5 activation and used to normalize the results.

Anti CD28 5 ug/mL soluble and anti CD3, 10 ug/mL plated were used to stimulate OTII CD4+ T cells (5×104 cells/well, 96 well plate) for 3 days. IFNγ were measured by ELISA (BD Bioscience).

Results

pUDCA functions as a drug carrier but is also intrinsically therapeutic. With pUDCA_{INS}, blood glucose control is thus a convolution of direct insulin provision to the blood pool as well as intrinsic therapeutic benefit as a result of strong ligation of BA receptors. The extent to which insulin is made bioavailable from the material in both the pancreas and the blood pool is consistent with earlier studies of biodistribution of orally ingested dye loaded pUDCA, insulin provisioned by pUDCA first appears higher in the pancreas, then naturally flows to the blood pool, highlighting the notion that pUDCA mediated delivery of insulin occurs in a physiologically relevant manner where it originates from the pancreas then made available in the blood. At 4, 8, 24 h post oral gavage of pUDCA_{INS}, increased pancreatic and blood insulin from pUDCA compared to delivery via PLGA or injection of soluble insulin was observed. See FIGS. 9A-9C.

Second, pUDCA's inherent therapeutic character is underscored by the fact that, with no encapsulated insulin, it increased endogenous glucagon-like peptide (GLP-1) secretion and insulin production (FIGS. 10A, 10B). GLP-1, an incretin, has insulinotropic activity and can decrease blood sugar levels by promoting endogenous insulin secretion. The fact that GLP-1 secretion from intestinal enteroendocrine L cells is abrogated upon blockade of the TGR5 receptor in vivo after pUDCA ingestion emphasizes the role of pUDCA in binding to TGR5 and induction of therapeutic effects that go beyond insulin delivery alone. Indeed, beyond the metabolic control of insulin, binding to TGR5 resulted in T cell frequency immunomodulation with a reduction of CD44+CD8+ T cell frequency and an increase in Tregs in pancreatic lymph nodes of animals treated with pUDCA, which is consistent with T1D prevention studies.

Both UDCA and pUDCA were able to induce insulin secretion from pancreatic β cells than did PLGA particles (FIG. 4M)

A reduction in interferon gamma (IFNγ) was also observed upon exposure of T cells to pUDCA followed by engagement of dendritic cells presenting antigens (FIG. 4D), or upon directly treating the CD4+ T cells with pUDCA and stimulated with anti-CD3 and anti-CD28 (FIG. 4N) and phenotypic skewing of macrophages from M1 to M2 (FIG. 11B). The anti-inflammatory function of pUDCA thus spans multiple immunological mechanisms ranging from innate to adaptive immunity and is consistent with prior reports exemplifying the various therapeutic and immunomodulatory facets of the monomer UDCA in different disease states. pUDCA is, however, a significantly potent format of UDCA and as such introduces mechanisms and modalities of function not possible with UDCA alone as demonstrated in FIGS. 10A-10C, 11A-11B.

Unless defined otherwise, all technical and scientific terms used herein have the same meanings as commonly understood by one of skill in the art to which the disclosed invention belongs. Publications cited herein and the materials for which they are cited are specifically incorporated by reference. Those skilled in the art will recognize, or be able to ascertain using no more than routine experimentation, many equivalents to the specific embodiments of the invention described herein. Such equivalents are intended to be encompassed by the following claims.

We claim:

1. An anti-inflammatory formulation comprising an effective amount of nanoparticles comprising bile acid ester polymers having a molecular weight between about 8000 and 240,000 Daltons (Da), wherein the nanoparticles do not comprise therapeutic or prophylactic agent.

2. The formulation of claim 1, wherein the bile acid ester polymers have a molecular weight between about 800 and 20,000 Da.

3. The formulation of claim 2, wherein the bile acid ester polymers are pUDCA having a molecular weight between about 800 and 5,000 Da.

4. The formulation of claim 1 wherein the nanoparticles having diameters between diameters between 60 nm and 600 nm, more preferably between 100 nm and 400 nm, with a typical average geometric diameter of 350 nm.

5. The formulation of claim 1, wherein the bile acid ester polymers are selected from the group consisting of polymeric ursodeoxycholic acid (pUDCA), polymeric lithocholic acid (pLCA), polymeric deoxycholic acid (pDCA), polymeric chenodeoxycholic acid (pCDCA), and polymeric cholic acid (pCA).

6. The formulation of claim 1, wherein the bile acid polymers are pUDCA having as shown in Formula VII: 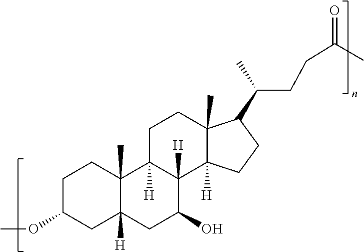
wherein n is a number between 2 and 20.

7. The formulation of claim 1, wherein the bile acid polymers form a surface on the nanoparticles comprising between 100 and 5000 bile acid monomers.

8. The formulation of claim 1, wherein the bile acid polymers form a surface on the nanoparticles comprising between 100 and 3000 bile acid monomers.

9. The formulation of claim 1, wherein the bile acid polymers are linear and/or branched polymers.

10. The formulation of claim 1, wherein the nanoparticles have at least 1.5 fold greater affinity to bile acid receptors than respective monomers forming the bile acid polymers.

11. A method of treating an inflammatory, autoimmune disease, or metabolic disease in a subject comprising administering to the subject an effective amount of the formulation of claim 1.

12. The method of claim 10, wherein the nanoparticles are administered orally.

13. The method of claim 11, wherein the nanoparticles distribute to internal organs selected from the group consisting of heart, kidneys, spleen, lungs, colon, liver, and pancreas.

14. The method of claim 10, wherein the inflammatory or autoimmune disease is selected from the group consisting of type 1 diabetes, type 2 diabetes, pancreatitis, hepatitis, cirrhosis, inflammatory bowel disease, colitis, systemic lupus erythematous, and rheumatoid arthritis.

15. The method of claim 10 wherein the subject is pre-diabetic with elevated blood glucose.

16. The method of claim 10 wherein the subject has diabetes.

17. The method of claim 10 wherein the formulation is administered to provide weight control.

18. The method of claim 10, wherein the effective amount of the formulation comprises between about 0.1 mg/kg and 1000 mg/kg nanoparticles.

19. The method of claim 10, wherein the formulation is administered for a period of at least one week, at least two weeks, or at least three weeks.

20. The method of claim 10, wherein the formulation is administered three times a week, two times a week, or once a day.

21. The method of claim 10, wherein the subject maintains normal blood glucose for at least about three days, about five days, about one week, about two weeks, about one month, or more, following cessation of administering the formulation of claim 1.

22. The method of claim 18, wherein the method increases the number of regulatory T cells (Treg) in the subject relative to a control.
